(19) United States
(12) Patent Application Publication
(10) Pub. No.: US 2024/0002229 A1
(43) Pub. Date: Jan. 4, 2024

(54) DEVICE FOR ENHANCING REACTION POTENTIAL OF OXIDIZING AGENTS

(71) Applicant: Bis Science LLC, Fort Worth, TX (US)

(72) Inventors: Paul DABNEY, Georgetown, TX (US); Marc W. GUNDERSON, Fort Worth, TX (US)

(73) Assignee: Bis Science LLC, Fort Worth, TX (US)

(21) Appl. No.: 18/346,304

(22) Filed: Jul. 3, 2023

Related U.S. Application Data
(60) Provisional application No. 63/357,739, filed on Jul. 1, 2022.

(51) Int. Cl. C01B 15/027; B01J 19/12; B01J 19/08; B01J 19/00; A61L 2/26; A61L 2/22; A61L 2/20; A61L 2/18

(52) U.S. Cl. CPC C01B 15/027; B01J 19/121; B01J 19/123; B01J 19/127; B01J 19/125; B01J 19/082; B01J 19/0013; A61L 2/26; A61L 2/22; A61L 2/208; A61L 2/186; B01J 2219/0801; B01J 2219/1203; B01J 2219/0877; A61L 2101/02

(57) ABSTRACT

Methods, systems, and apparatuses for producing one or more of photon enhanced oxidizing agents, trioxygen, hydrogen and its ions, oxygen and its ions, ROS and electronically modified oxygen derivatives from oxidizing agents that are exposed to photon emissions at a wavelength in a range of 0.01 nm to 845 nm, wherein wavelengths that photo-dissociate trioxygen may be excluded. The methods, systems and apparatuses enhance the effectiveness of photo-oxidation, photocatalytic, and/or photochemical reactions or a combination of these reactions.

### CROSS-REFERENCE TO RELATED APPLICATIONS

This application claims priority to U.S. Provisional Patent Application No. 63/357,739, filed Jul. 1, 2022, the entire contents of which are incorporated herein by reference.

### BACKGROUND

Photon-enhanced thermionic emission (PETE) combines the quantum and thermal processes obtained from a reaction into a single physical process to take simultaneous advantage of photons and of the available thermal energy of the generated phonons. Thermionic emission is the liberation of electrons by virtue of its temperature. Releasing of energy supplied by phonons. This occurs because the thermal energy given to the charge carrier overcomes the work function of the material. The charge carriers can be electrons, or ions. Charge carriers are particles or holes that freely move within a material and carry an electric charge. In most electric circuits and electric devices, the charge carriers are negatively charged electrons that move under the influence of a voltage to create an electric current. However, most circuitry is designed in terms of conventional current, which involves positive charges that move in the opposite direction of electrons. Other than electrons and positively charged particles, holes are also charge carriers. Holes are empty valence electron orbitals, and as such, they represent an electron deficiency that can move freely within a material. This disclosure utilizes photons and phonons in a unique device and method to achieve photon augmented oxidizing agents (PAOA) that can be utilized to generate a self-sustaining circuit of reactions.

### SUMMARY

Various embodiments include methods and steps of applying at least one oxidizing agent to a target or a substance or area to be treated, applying photon emissions at one or more wavelengths in a range from less than 0.01 nm through 845 nm (845 nm, the upper wavelength that photolyzes oxygen to oxygen bonds in hydrogen peroxide, 0.01 nm the lower limit of x-ray photons) to the oxidizing agent, the target, and/or the substance or area to be treated, where wavelengths that photo-dissociate trioxygen may be excluded, and performing an oxidizing reaction between the at least one oxidizing agent and the target and/or area or substance to be treated, which produces photo-oxidation reaction products (PETE) reactions, photocatalytic reaction products, photochemical reaction products, and/or photochemical combined, and/or a combination of these reactions and their products. The resulting reactions occur where the photo oxidation reaction products, photocatalytic reaction products, photochemical reaction products, and/or a combination of these reactions generates a self-sustaining circuit of reactions. This generates at least one of x-ray photons, hydrons, trioxygen, hydrogen and its ions, oxygen and its ions, hydroxyl radical, ROS, trioxidane, and electronically modified oxygen derivatives (EMODS or EMODs).

Various embodiments include a reaction area, in which at least one oxidizing agent functions together with photon emissions of from 0.01 nm through 845 nm (845 nm, the upper wavelength that photolyzes oxygen to oxygen bonds in hydrogen peroxide, 0.01 nm the lower limit of x-ray photons) to enhance the reaction potential of an oxidizing agent. This creates an oxidizing agent with an increased reaction potential that's displayed when performing an ionization reaction and/or an oxidation reaction. The device contains at least one oxidizing agent introducing component for applying the at least one oxidizing agent to the target and/or area or substance to be treated, and at least one photon emitting component for creating the photon emissions. In an embodiment, the described reactions of the device can take place in many areas. The reactions can be performed in a container where the photon emissions are directed at the oxidizing agent generating photon enhanced oxidizing agents that exhibit increased reaction potential. The photon enhanced oxidizing agents (PEOA) can be applied to a target to be treated by the device by a mister, sprayer, pump, fogger or any other suitable means. This target can be a substance or an area where the reactions of this disclosure are desired. The reactions can be performed in ambient air where an oxidizing agent is sprayed, misted, fogged or otherwise applied to the air then the airborne oxidizing agent can be exposed to photons to generate the PEOA by the device and its methods. The reaction area can be described as a location where the oxidizing agent is exposed by the device to the generated photons. These photons can be generated in an manner, including x-ray generators, LEDs, bulbs, arc lights, plasma lights, lasers, or any other suitable method. These examples of areas and photon generators are not meant to be all inclusive, but are meant to serve as examples of areas where the reactions may occur and examples of methods or devices that may generate photons for the reactions. The described methods may produce precipitates in air, liquid, plasma, and solids as a result of the reactions generated by the device and methods of the embodiments. These products may have desired applications or uses. Precipitates produced as a result of the described methods may be separated and collected from reactants by the device. This separation and collection may involve centrifugation, filtration or any other suitable means. The embodiments generate the reactions resulting from the interactions between oxidizing agents and photons of certain wavelengths and frequency. The oxidizing agent or oxidizing agents may be introduced to the reaction area by a pump, mist, fog, spray, dripline, or any other suitable component. This oxidizing agent introducing component of this device functions so that the photon emitting component exposes the oxidizing agent or oxidizing agents to the photons either before the oxidizing agent is applied to a target or while the oxidizing agent is applied to a target or after an oxidizing agent is applied to a target or a combination of these. A target may be an area or substance or place where the described reaction methods of the embodiments are to react or take place.

Oxidizing agents that have been exposed to photon emissions can subsequently be placed in pressure vessels of this device where the evolving gases are not allowed to escape. If these pressure vessels associated with this device reflect and scatter x-ray photons, then endogenous generated x-ray photons perpetuate the described self-sustaining reaction generated by the embodiments. This further confirms the new art described herein. A self-sustained reaction that generates EMODs, ROS, hydrogen and its ions, oxygen and its ions, hydrons, trioxidane, and other free radicals is created and exists until one of the reactants is depleted. The elevated reactivity of the photon enhanced oxidizing agent generated by the embodiments can be demonstrated for extended periods of time. The more x-ray reflective the container holding the photon augmented oxidizing agent, the greater the self-sustained reaction that is created.

### BRIEF DESCRIPTION OF THE DRAWINGS

Non-limiting and non-exhaustive embodiments of the disclosure are described with reference to the following figures and detailed description.

FIG. 1 is an exemplary diagram showing diffusion of particles;

FIG. 2 is an exemplary diagram showing that a reaction can occur from a reactant molecule via an intermediate such as hydroperoxyl to form a trioxygen molecule;

FIG. 3 is an exemplary diagram showing a Geiger counter reading with experimental results; and

FIG. 4 is an exemplary diagram showing a Geiger counter reading with experimental results.

### DETAILED DESCRIPTION OF THE EMBODIMENTS

Aspects of the present disclosure are disclosed in the following description and related drawings, diagrams and pictures directed to specific embodiments. Alternate embodiments may be devised without departing from the spirit or the scope of the disclosure. Additionally, well-known elements of exemplary embodiments will not be described in detail or will be omitted so as not to obscure the relevant details of the disclosure.

As used herein, the word “exemplary” means “serving as an example, instance, or illustration.” The embodiments described herein are not limiting, but rather are exemplary only. The described embodiments are not necessarily to be construed as preferred or advantageous over other embodiments. Moreover, the terms “embodiment or “embodiments” do not require that all embodiments of the disclosure include the discussed feature, advantage, or mode of operation.

As used herein, the terms “and/or” and “and or” as used herein means that two or more elements are to be taken together or individually. Thus, “A and/or B” and “A and or B” cover embodiments having element A alone, element B alone, or elements A and B taken together.

Generated gases created in the reactions of the device may be vented by any appropriate means desired or these gases may be retained. This venting may be a means to control or modulate the reaction. As an example, the produced gases may be totally captured to preserve the highest reaction potential, or the generated gases may be fully vented if the reaction potential needs to be reduced or halted.

According to various embodiments, the various micron-sized droplets created by the embodiments evaporate at selected rates, depending on application needs. In some embodiments, small size particles are selected, and they are sized so that they completely evaporate into the air before reaching most surfaces. This near 100% evaporation rate achieves near 100% chemical efficiency. In some embodiments, the particle fall rate is calculated based on density, size, and mass of the particle as well as the density of the air or gas it is placed in. Humidity also influences the fall rate outcome because at a low humidity a particle will tend to evaporate faster and lose size and mass as it remains air borne. These factors, when based on the embodiments, enable various embodiments of a selected size micron fog microbial suppression system, and/or agglomeration system, and/or bleaching system, or other applicable system to utilize an extremely low volume and low concentration of a photon enhanced oxidizing agent solution. These solutions generate endogenous x-ray photons that create a self-sustaining circuit of reactions. This self-sustaining circuit of reactions can be intensified by placing the photon enhanced oxidizing agent in a container or area where the endogenous x-ray photons can be reflected back into the PEOA. By reflecting these endogenous x-ray photons back into the PEOA, they are available to further ionize the PEOA solution. This device creates a PEOA solution that is more reactive and reactive longer than an oxidizing agent solution that is not enhanced with photons as described in the embodiments.

According to various embodiments, the photon enhanced oxidizing agent (PEOA) solution is deposited into a volume of liquid, plasma, air, or gas, or other suitable medium. In various embodiments, this is done through an existing HVAC system, a fogging device, a sprayer, a mister, an injector, a dropper, a spray can, an aerial spraying device, crop dusting, or other suitable devices.

The embodiments are further directed to a device and system for progressive regression of Colony Forming Units (CFUs) from the continuous presence of a photon enhanced microbial suppression system utilizing photon enhanced oxidizing agents. Embodiments provide a decontamination system that includes a photon enhanced microbial suppression system solution, the PEOA, and its effects on substances that it contacts. Various embodiments utilize a MPA, PETE and a photon augmented oxidizing agent containing microbial suppression device and system that includes particle size considerations for controlled dispersion and addresses agglomeration of inactivated microbes and other precipitates in a multi-faceted technology described by the device and system. In various embodiments, this combination provides a way for decontaminating areas, structures, food, liquids, animals, animal fluids, plants, buildings, pipelines, homes, offices, indoors and outdoors. Some embodiments feature low chemical concentrations made more effective with the combination of the photon enhanced oxidizing agent microbial suppression device and system, so that there are reduced or no harmful effects on humans or animals or plants when administered at these low concentrations, and so exposure to the PEOA agents can be on going, constant, or nearly constant. This low concentration contrasts sharply with oxidizing agent solutions that have not been augmented with photon emissions as described herein.

Use for Blood Dissociation

Various embodiments include a decontamination device and system whereby blood components go through the described agglomeration process whereby photon enhanced oxidizing agents are added to the blood causing dissociation of the blood into constituent components allowing for these components to be used for their water value and nutritional value and other desired purposes.

In some embodiments, a photon enhanced oxidizing agent, produced by the embodiments, is added to a substance (target) for antimicrobial purposes. In some embodiments, the effect of the photon emissions takes place at a certain time or place relative to the desired outcome of the reaction associated with the described methods. In these embodiments, the photon emissions will not be applied to the oxidizing agent/target mixture until such time as the photon enhanced reaction is desired to take place. In other instances, the photon emissions are applied to the oxidizing agent before it is applied to the target/mixture to be treated.

According to various embodiments, in techniques of sample processing, the animal fluids, blood, blood cells, microbes, and other organic matter of interest are first separated from the majority of substances by dissociation, agglomeration, and/or extraction methods when combined with oxidizing agents that have been exposed to photon and phonon emissions (PETE) from 0.01 nm through 845 nm. In various embodiments, extraction is performed in liquid phase or in a solid phase. In other embodiments, gross extraction of larger particles is sequenced with extraction methods processing progressively smaller units until the desired resolution is obtained. Various embodiments allow for this process to be accomplished by photon emissions applied to oxidizing agent solutions creating a PEOA.

In various embodiments, a photon emission enhanced antimicrobial oxidizing agent solution is applied to air via a HVAC system or other suitable means. In some embodiments, a small micron (less than 20 microns droplet size) mist or fog containing photon enhanced microbial suppression system is selected to utilize an extremely low volume and low concentration of a photon enhanced antimicrobial oxidizing agent solution into a volume of air or gas. In various embodiments, a 6-10 micron droplet size, 2-4 micron droplet size, or a sub 2 micron droplet size mist or fog of a photon enhanced oxidizing agent microbial suppression system is selected. The selected droplet size is selected based on the desired fall rate of the PAOA through the ambient air. Different air qualities may be better affected by different particle sizes of PAOA.

HVAC Applications

According to various embodiments, this is done through an existing HVAC system utilizing an electrostatic fog, fogging, misting, spraying, sprinkling, diffuser, atomizer, or other suitable device. In some embodiments, the application device includes one or more of an aerosolizing nozzles producing a small micron dry fog, an air compressor to push the solution through the nozzle at the desired rate, a metering pump to dispense the solution at a rate that will give the desired concentration in ambient air, and a control system to regulate and monitor the application of the solution. In some embodiments, a small micron dry fog photon augmented oxidizing agent microbial suppression system exhibits such a slow particle fall rate that when it is combined with the simultaneous evaporation of these particles, a concentration of PEOA gas vapor is created and maintained of the photon enhanced antimicrobial agent in the ambient air serviced by the HVAC system. This can also be referred to as the target. A progressive regression of CFUs from the continuous presence of the small micron dry fog microbial suppression system provides, in the ambient air, a decontamination system of air and surfaces that the small micron dry fog microbial suppression system solution contacts.

This demonstrates another, very different application of the technology in the present embodiments. The photon enhanced EOA antimicrobial and agglomeration effects, when used in HVAC systems as described, can be modulated by utilizing x-ray reflective containers or areas where the PEOA is deployed as previously described in the methods of this invention. These x-ray reflective containers or areas allow the endogenous generated x-ray photons to remain available to react with the PEOA.

In an example an embodiment of a system for progressive reduction of the microbial count in ambient air, a room with 1,000,000 colony forming units (CFUs) is equipped with a HVAC system that incorporates a device and system performing the methods as disclosed herein. A small micron PEOA antimicrobial dry fog is administered into the ambient air through an existing HVAC system and device at a concentration of less than 1 part per million. This low concentration PEOA causes a reduction in the microbial CFUs as the small micron PEOA dry fog slowly settles through the air killing microbial CFUs at a rate of about 20%.

Container

This effect can be modulated in the device and system by selecting a container or area that reflects the applied photons back into the generated photon enhanced oxidizing agent if desired.

Bleaching Method

An example of the described reaction used as a bleaching method can be illustrated with the preparation of wood pulp as used in paper manufacturing. One of the largest volume uses of hydrogen peroxide worldwide is pulp bleaching in the paper industry. Hydrogen peroxide is also used to increase the brightness of deinked pulp. The bleaching methods are similar for mechanical pulp in which the goal is to make the fibers brighter. By using various embodiments of the device and methods and the associated reactions described herein and augmenting the hydrogen peroxide with photons with a wavelength of 0.01 nm through 845 nm, (845 nm, the upper wavelength that photolyzes oxygen to oxygen bonds in hydrogen peroxide)(0.01 nm the lower limit of x-ray photons), a synergistic reaction takes place generating ROS, EMODs, Hydrogen and its ions, beta particles, hydrons, x-ray photons, oxygen and its ions and other free radicals and also produces photo-oxidation products, photocatalytic products, and/or photochemical products by photon absorption of the oxidizing agent and target, wherein the produced photo oxidation products, photocatalytic products, and/or photochemical products cause a greater bleaching result when compared with the same concentration of un-augmented oxidizing agent in the bleaching process. The device and methods described herein allow for the same bleaching effect with a lower concentration and/or volume of photon augmented oxidizing agent than un-augmented oxidizing agent. According to various embodiments, the self-sustaining circuit of reactions generated with PEOA, MPA, and PETE permits a device utilizing reactions that have not been described or utilized with a bleaching reaction previously. In addition, this reaction can be modulated by altering the x-ray photon reflectiveness of the container or area of the described reaction associated with the displayed device.

The device and system generates a self-sustaining circuit of reactions that permits an enhanced reaction that has not been described or utilized when contrasted with common reactions currently utilized in industries like the petroleum/petrochemical industries. The device's PEOA and the endogenous generated x-ray photons and endogenous generated beta particles generate a greater reactive potential by creating more ROS, EMODs, hydrogen and its ions, oxygen and its ions, beta particles, hydrons, x-ray photons and other free radicals when compared to un-augmented hydrogen peroxide that is currently used. The enhanced effect of PEOA can be modulated by altering the x-ray photon reflectiveness in containers or areas where this reaction takes place.

Sensors

To monitor the synergistic reaction described in the embodiments, various embodiments include at least one or more sensors or other devices to indicate, detect, or inform of one or more of the following properties of the target or storage or environment: pH, oxidation and reduction potential, electrical potential, temperature, salinity, density, trioxygen concentration, oxygen concentration, hydrogen concentration, oxidizing agent concentration, flow rate, microbial content, presence or absence of bacterial species, presence or absence of corrosive metabolites or otherwise corrosive substance, identification of a gas, presence or absence of an aqueous environment, presence or absence of high, low, or otherwise concentration of bacterial or non-bacterial, biomass or non-biomass, microbial content, or location of biofilms may be used. This list is not all inclusive but is meant to provide examples of sensors and other devices that may be used singularly or in multiples. According to various embodiments, these sensors may be used to help regulate the reactions described herein.

According to various embodiments of the device and system, the photon generating apparatus used in the methods described herein is located in or adjacent to the oxidizing agent to be enhanced. In some instances, the photon generating apparatus is located further from the oxidizing agent and methods of transmission of the photons are utilized. These methods of transmission include fiber optics, reflective materials, and other conductive media.

According to various embodiments of the device and system, flocculants are added to the reactions described herein before, during, or after the photon enhanced oxidizing agent is applied to the target where the described reaction is to take place. In some embodiments, flocculants are added before the reaction to remove substances that are not desired to undergo the described reaction. In other embodiments, the flocculant is added during the reaction or after the reaction depending on the desired outcome and use of the precipitated substance.

In various embodiments, the oxidizing agents are exposed to multiple frequencies of photon emission and multiple exposures of photon emission. In embodiments, the photons are supplied to the oxidizing agents continuously or in bursts or pulses. A continuous photon emission could be, for example, from a light emitting diode suspended in a container of an oxidizing agent emitting a constant dose of photons. Bursts or pulses of photon emission could be utilized to rapidly enhance an oxidizing agent with 0.01 nm-845 nm (845 nm, the upper wavelength that photolyzes oxygen to oxygen bonds in hydrogen peroxide) (0.01 nm the lower limit of x-ray photons), photon, for example from a high intensity laser where the high intensity bursts or pulses may be only seconds in duration, but these bursts or pulses could provide the same dose of photon emissions as a long duration continuous photon emission that was at a low dose, where dose is defined as intensity of the photon emission times the time of application.

In various embodiments, the photon wavelength in a range of 0.01 nm to 845 nm (845 nm, the upper wavelength that photolyzes oxygen to oxygen bonds in hydrogen peroxide) (0.01 nm the lower limit of x-ray photons), is produced from a variety of sources such as x-ray generators, LEDs, lasers, natural light, electromagnetic radiation, arc lamps and other suitable sources. The list of radiation producing sources is not meant to limit sources to those listed but to serve as an example.

According to various embodiments of the methods, the reactants contain enzymes, stabilizers, or other substances that affect the overall reaction rate.

According to various embodiments, a device and method for enhancing the effectiveness of products generated from ionization reactions, photo-oxidation reactions, photocatalytic reactions, and/or photochemical reactions or a combination of these reactions is provided. The reaction products contain one or more of reactive nitrogen species, x-ray photons, hydrogen and/or its isotopes, oxygen and/or its isotopes, beta particles, hydrons, electronically modified oxygen derivatives, reactive oxygen species, trioxygen, and other free radicals. Various embodiments of the device and method include: applying at least one oxidizing agent to a target or a substance to be treated; applying photon emissions at one or more wavelength in a range of 0.01 nm through 845 nm (845 nm, the upper wavelength that photolyzes oxygen to oxygen bonds in hydrogen peroxide)(0.01 nm is the lower wavelength range for x-rays) to the oxidizing agent, the target, and/or the substance to be treated, wherein wavelengths that photo-dissociate trioxygen may be excluded; and performing an oxidizing reaction between the at least one oxidizing agent and the target and/or substance to be treated, which produces the products, and/or photochemical or a combination of these reaction products, wherein the ionization reaction products, photo oxidation reaction products, photocatalytic reaction products, and/or photochemical combined with photocatalytic reaction products generate at least one of x-ray photons, trioxygen, hydrogen and its ions, oxygen and its ions, beta particles, hydrons, hydroxyl radical, and electronically modified oxygen derivatives and other free radicals.

In various embodiments, the photon emissions are applied by a photon emission source selected from an x-ray generator, electromagnetic radiation emitting bulb, a light emitting diode, an electrical ion generator or a laser or any other suitable means of generating photons of the required wavelength or wavelengths.

In various embodiments, the photon emissions are applied directly or indirectly to the oxidizing agent, and/or the target, and/or the substance or area to be treated.

In various embodiments, the at least one oxidizing agent is applied to the target or the substance or area to be treated with an oxidizing agent dispenser selected from a pump, mister, fogger, atomizer, diffuser, electrostatic sprayer, or other suitable device that dispenses the oxidizing agent in a desired particle size.

Various embodiments further include applying additional reactants at various stages to aid the oxidizing reaction, wherein the additional reactants are selected from enzymes, catalysts, stabilizers, and flocculants or other suitable agents.

In various embodiments, the oxidation reaction occurs in a sealed container whereby gases created by the oxidation reaction are not allowed to escape.

According to various embodiments, a device and system is configured to perform a method for enhancing the effectiveness of products generated from ionization reactions, photo-oxidation reactions, photocatalytic reactions, photochemical reactions, and/or a combination of these reactions. The device and system includes: a reaction area, in which the at least one oxidizing agent functions together with photon emissions to perform the ionization and/or oxidation reactions, so that products of the ionization and/or oxidation reaction can be collected and separated at any time during the reactions; at least one oxidizing agent introducing component for applying the at least one oxidizing agent to the target and/or substance or area to be treated; and at least one photon emitting component for creating the photon emissions.

Various embodiments of the device and system further include one or more sensors or other devices to indicate, detect, or inform of one or more of the following properties of the reactants, target or storage or environment: pH, temperature, salinity, x-ray radiation, gamma radiation, pressure, oxidation and reduction potential, density, trioxygen concentration, oxygen concentration, hydron concentration, gamma ray concentration, beta particle concentration, hydrogen concentration, oxidizing agent concentration, flow rate, microbial content, presence or absence of bacterial species, presence or absence of corrosive metabolites or otherwise corrosive substance, identification of a gas, presence or absence of an aqueous environment, presence or absence of high, low, or otherwise concentration of bacteria or non-bacteria, biomass or non-biomass, or microbial content, and location of biofilms.

In various embodiments of the device and system, the at least one photon emitting component emits, delivers, produces, or otherwise facilitates photon emissions in a range from 0.01 nanometers to 845 nanometers, independently, simultaneous, continuously, or intermittently, and the at least one photon emitting component is suspended, adjacent to, inside of, surrounding, or associated with a container, structure, area of the at least one oxidizing agent, the target, and/or substance to be treated, and/or supported in a target container, and wherein the at least one photon emitting component is or is not physically close to the at least one oxidizing agent, the target, and/or the substance or area to be treated.

In various embodiments of the device and system, the at least one photon emitting component adjusts one or more of the photon emission wavelengths, frequency, intensity, duration, or location relative to the target and/or substance or area to be treated on the basis of any one or more of the density and light absorbing or reflection or scattering quality of the target and/or substance or area to be treated, the size, shape, or composition of the reaction area, conditions or properties of the environment, whether the target and/or substance or area to be treated is under aerobic or anaerobic conditions, pH, temperature, or salinity of the target and/or substance or area to be treated, consortium or population characteristics of any organisms or micro-organisms present in the target and/or substance or area to be treated, microbial content of the target and/or substance or area to be treated, and the microbial content of any biofilm present in the target and/or substance or area to be treated.

Aspects of the embodiments are disclosed in the following description and related drawings diagrams and pictures. Alternate embodiments may be devised without departing from the spirit or the scope of the disclosure. Additionally, well-known elements of exemplary embodiments will not be described in detail or will be omitted so as not to obscure the relevant details of the disclosure.

As used herein, the word “exemplary” means “serving as an example, instance, or illustration.” The embodiments described herein are not limiting, but rather are exemplary only. The described embodiments are not necessarily to be construed as preferred or advantageous over other embodiments. Moreover, the terms “embodiment or “embodiments” do not require that all embodiments include the discussed feature, advantage, or mode of operation.

In the device, methods and systems disclosed herein, methods of utilizing both homogeneous and heterogeneous photocatalytic (PCA) reactions are described. By utilizing both types of PCA in the described device and methods, a photon augmented self-sustaining reaction is produced resulting in generated electronically modified oxygen derivatives, reactive oxygen species, hydrogen and its ions, oxygen and its ions, hydrons, trioxidane, and other free radicals that are continuously produced as long as reactants are present. Generated gases created in the reactions of the device may be vented by any appropriate means desired or these gases may be retained. This venting may be a means to control or modulate the reaction. As an example, the produced gases may be totally captured to preserve the highest reaction potential, or the generated gases may be fully vented if the reaction potential needs to be reduced or halted. Trioxygen is one of the potential photocatalysts generated by the described reactions of the device in this embodiment. Trioxygen and the endogenous x-ray photons produced results in an increased efficacy and a shelf life of increased and sustainable reactivity in the PEOA when compared and contrasted with oxidizing agents that are not exposed and enhanced with exogenous photon emissions. Research has found that trioxidane is one of the active ingredients responsible for the antimicrobial properties of the ozone/hydrogen peroxide mix. Because these two compounds are present in biological systems as well it is argued that an antibody in the human body can generate trioxidane as a powerful oxidant against invading bacteria. Trioxidane can be obtained in small, but detectable, amounts in reactions of ozone and hydrogen peroxide.

As used herein, the terms “and/or” and “and or” as used herein means that two or more elements are to be taken together or individually. Thus, “A and/or B” and “A and or B” cover embodiments having element A alone, element B alone, or elements A and B taken together.

Ionizing radiation consists of subatomic particles or electromagnetic waves that have sufficient energy to ionize atoms or molecules by detaching electrons from them. Gamma rays, x-rays, and some parts of the ultraviolet part of the electromagnetic spectrum are commonly considered ionizing radiation, whereas visible light, nearly all types of laser light, infrared, microwaves, and radio waves are commonly considered non-ionizing radiation. The boundary between ionizing and non-ionizing radiation is not sharply defined because different molecules and atoms ionize at different energies. Photons may be called x-rays if they are produced by electron interactions, and they are of the appropriate wavelengths. An x-ray photon has a wavelength of 0.01 to 10 nanometers, with a frequency of 3×10¹⁶ Hz to 3×10¹⁹ Hz. It possesses enough energy (100 eV to 100 keV) to disrupt molecular bonds and ionize atoms making it, by definition, ionizing radiation. The energy of ionizing radiation is between 10 electronvolts (eV) and 33 eV. Even though photons are electrically neutral, they can ionize atoms indirectly through the photoelectric effect and the Compton effect. Either of those interactions will cause the ejection of an electron from an atom at relativistic speeds, turning that electron into a beta particle (secondary beta particle) that will ionize other atoms. Beta particles (β) are high-energy, high-speed electrons (β−) or positrons (β+) that are ejected from an atom. As they have a small mass and can be released with high energy, they can reach relativistic speeds (close to the speed of light). In a photon enhanced heterogeneous system, when the two phases each constitute a significant fraction of the total mass, the ionizing energy is absorbed significantly by both phases. After the absorption of a high-energy photon, a high energy Compton electron is ejected. This electron induces a large number of secondary electrons of energies in the 100 eV range. Since most of the ionized atoms in the embodiments are due to the secondary beta particles, photons endogenously produced within the methods described herein are indirect ionizing radiation. Radiated photons are called gamma rays if they are produced by a nuclear reaction, subatomic particle decay, or radioactive decay within the nucleus. They are called x-rays if produced outside the nucleus. An x-ray is a packet of electromagnetic energy (photon) that originates from the electron cloud of an atom. This is generally caused by energy changes in an electron, when it moves from a higher energy level to a lower one, causing the excess energy to be released. X-rays are similar to gamma rays in many respects however the main difference is the way they are produced. X-rays are produced by electrons external to the nucleus. The generic term “photon” is used to describe both. X-rays have a lower energy than gamma rays. Photoelectric absorption is the dominant mechanism of interaction in organic materials for photon energies below 100 keV. At energies beyond 100 keV, photons ionize matter increasingly through the Compton effect, and then indirectly through pair production at energies beyond 5 MeV. In a scattering event, the photon transfers energy to an electron, and then continues on its path in a different direction and with reduced energy. The x-ray photons produced in this manner range in energy from near zero up to the energy of the electrons. An incoming photon may also collide with an atom in the target, kicking out an electron and leaving a vacancy in one of the atom's electron shells. Another electron may fill the vacancy and in so doing release an X-ray photon of a specific energy. Bremsstrahlung radiation is electromagnetic radiation produced by the deceleration of a charged particle when deflected by another charged particle. The moving particle loses kinetic energy, which is converted into radiation (photons), thus satisfying the law of conservation of energy. X-rays are emitted as the electrons slow down (decelerate). The output spectrum consists of a continuous spectrum of x-rays, with additional sharp peaks at certain energies. The continuous spectrum is due to bremsstrahlung, while the sharp peaks are characteristic x-rays associated with the atoms in the target. Bremsstrahlung radiation is a type of “secondary radiation”, in that it is produced as a result of stopping (or slowing) of the primary photon radiation. Ionization of molecules can lead to radiolysis (breaking chemical bonds), and formation of highly reactive free radicals. These free radicals may then react chemically with neighboring materials even after the original radiation has stopped. Ionizing radiation can also accelerate existing chemical reactions by contributing to the activation energy required for the reaction. Compton scattering is the scattering of a photon after an interaction with a charged particle, usually an electron. If it results in a decrease in energy of the photon, it is called the Compton effect. Part of the energy of the photon is transferred to the recoiling electron. Inverse Compton Scattering occurs when a charged particle transfers part of its energy to a photon. As given by Compton, the explanation of the Compton shift is that in the target material valence electrons are loosely bound in the atoms and behave like free electrons. Compton assumed that the incident x-ray radiation is a stream of photons. An incoming photon in this stream collides with a valence electron in the target. During this collision, the incoming photon transfers some of its energy and momentum to the target electron and leaves the scene as a scattered photon. Simply, a photon that has lost some of its energy emerges as a photon with a lower frequency, or equivalently, with a longer wavelength. The discoveries described herein are utilized in the displayed device and generate PETE, exogenous applied photons, endogenous generated photons, the photoelectric effect and the Compton effect to excite electrons in target materials while generating electronically modified oxygen derivatives (EMODs) and reactive oxygen species (ROS), hydrogen and its ions, oxygen and its ions, hydrons and other free radicals. Reactive oxygen species (ROS) are highly reactive chemicals formed from O₂. Examples of ROS include peroxides, super oxides, hydroxyl radicals, trioxygen, singlet oxygen, and alpha oxygen. The reduction of molecular oxygen (O₂) produces superoxide(^{·}O⁻₂), which is the precursor to most other reactive oxygen species:

O₂+e⁻→^{·}O⁻₂

Dismutation of superoxide produces hydrogen peroxide (H₂O₂):

2H⁺+^{·}O⁻₂+^{·}O⁻₂→H₂O₂+O₂

Hydrogen peroxide in turn may be partially reduced, thus forming, hydrogen ions, hydroxide ions and hydroxyl radicals (^{·}OH), or fully reduced to water:

H₂O₂+e⁻→HO⁻+^{·}OH

2H⁺+2e⁻+H₂O₂→2H₂O

EMODs, ROS, hydrogen and its ions, oxygen and its ions, hydrons and other free radicals generated by the device and methods of the embodiments continue to react with target materials and/or target areas even after the application of exogenous photon application has stopped. If the reaction of EMODs, ROS, hydrogen and its ions, oxygen and its ions, free radicals, trioxidane, and endogenous x-ray photons with an oxidizing agent generates x-ray photons, a self-sustaining reaction can be created that further produces EOMDs, ROS, hydrogen and its ions, oxygen and its ions, hydrons and other free radicals. X-ray photons scattered by a set of atoms produce x-ray radiation in all directions, leading to interferences due to the coherent phase differences between the interatomic vectors that describe the relative position of atoms. In a molecule or in an aggregate of atoms, this effect is known as the effect of internal interference, while we refer to an external interference as the effect that occurs between molecules or aggregates. As mentioned previously, multi-proton absorption, MPA, contributes to the production of EMODs, ROS, hydrogen and its ions, oxygen and its ions, hydrons other free radicals and endogenous x-ray photons. MPA and its effects have been ignored or under appreciated by most other disclosures. The self-sustained circuit of reactions generated and employed by the device and methods displayed in the embodiments are unique in that the created reactions generate EMODs, ROS, hydrogen and its ions, oxygen and its ions, hydrons and free radicals, trioxidane, and endogenous x-ray photons at rates that previously have not been demonstrated.

Water absorbs UV radiation near 125 nm, exiting the 3a1 orbit and leading to dissociation into OH⁻ and H⁺. Through MPA, this dissociation can also be achieved by two or more photons at other nm wavelengths. This creates reactions and products, and embodiments, that have not been previously demonstrated, reported or understood. Multi-photon absorption (MPA) and two photon absorption (TPA) are terms used to describe a process in which an atom or molecule makes a single transition between two of its allowed energy levels by absorbing the energy from more than a single photon. This can generate ionization and oxidation of substances involved in the reactions releasing beta particles, endogenous x-ray photons, EMODs, ROS, hydrogen and its ions, oxygen and its ions, hydrons, trioxidane and other free radicals.

Chemi-excitation via oxidative stress by reactive oxygen species, electronically modified oxygen derivatives, hydrogen and its ions, oxygen and its ions, hydrons, trioxidane, and/or catalysis by enzymes is a common event in biomolecular systems. The embodiment relates to utilizing exogenous applied photons and endogenous generated photons that are applied to an oxidizing agent generating a synergistic chemi-excitation process that generates ROS, electronically modified oxygen derivatives (EMODs) hydrogen and its ions, oxygen and its ions, endogenous x-ray photons, beta particles, hydrons, trioxidane and other free radicals. According to various embodiments of the embodiments, such reactions may lead to the formation of triplet excited species such as trioxygen (ozone, O₃), and hydroxyl radicals, hydrons, trioxidane, and other free radicals. This process contributes to spontaneous biophoton emission. In further embodiments of this device, photon emission is increased by the generation of endogenous photons produced from the enhanced oxidizing agent that in turn generate ROS, EMOD, hydrogen and its ions, oxygen and its ions, beta particles, x-ray photons, hydrons and other free radicals such as hydroxyl radicals, hydroperoxides, singlet oxygen, hydrogen, superoxide, and others.

Electromagnetic radiation moves in a vacuum at a universal speed. This is the speed of light, c=30,000,000,000 centimeters per second (usually written in powers of ten, c=3×10¹⁰ cm/sec). The constant value of the speed of light in vacuum goes against our intuition: we would expect that high energy (short wavelength) radiation would move faster than low energy (long wavelength) radiation. We can consider light as a stream of minute packets of energy, photons and biophotons and generated phonons, which creates a pulsating electromagnetic disturbance. A single photon or biophoton differs from another photon or biophoton only by its energy. In empty space (vacuum), all photons and biophotons travel with the same speed or velocity.

Photons and biophotons are slowed down, generating phonons and/or interacting with atoms or molecules thereby releasing electrons and endogenous photons, when they interact with different media such as water, glass or even air. This slowing down accounts for the refraction or bending of light. Refraction is the bending of a wave when it enters a medium where its speed is different. The refraction of light when it passes from a fast medium to a slow medium bends the light ray toward the normal to the boundary between the two media. The amount of bending depends on the indices of refraction of the two media and is described quantitatively by Snell's Law. As the speed of light is reduced in the slower medium, the wavelength is shortened proportionately. The energy of the photon and biophotons is not changed, but the wavelength is. Different energy photons and biophotons are slowed by different amounts in glass or water or other substances; this leads to the dispersion of electromagnetic radiation. As used herein, greater intensity of light means that more photons were available to hit a target per second and more electrons could be ejected from a target, not that there was more energy per photon or biophoton.

The energy of the outgoing electrons depends on the frequency of photons. There are two predominant kinds of interactions through which photons deposit their energy—both are with electrons. In one type of interaction the photon loses all its energy; in the other, it loses a portion of its energy, and the remaining energy is scattered. The energy E of the incoming photons and biophotons is directly proportional to the frequency, which can be written as E=hf in which h is a constant. Max Planck first proposed this relationship between energy and frequency in 1900 as part of his study of the way in which heated solids emit radiation. In one example, the photoelectric (photon-electron) interaction, a photon transfers all its energy to an electron located in one of the atomic shells. The electron is then ejected from the atom by this energy and begins to pass through the surrounding matter. The electron rapidly loses its energy and moves only a relatively short distance from its original location. The photon's energy is deposited in the matter close to the site of the photoelectric interaction. The energy transfer is a two-step process. The photoelectric interaction in which the photon transfers its energy to the electron is the first step. The depositing of the energy in the surrounding matter by the electron is the second step. Photon-enhanced thermionic emission (PETE) and electrons are the two main types of elementary particles or excitations generated with photon reactions. MPA increases the photoelectric interactions described in the embodiments. MPA increases the amount of energy available to be deposited in the surrounding matter.

If the binding energy is more than the energy of the photon, a photoelectric interaction cannot occur. This interaction is possible only when the photon has sufficient energy to overcome (ionize) the binding energy and remove the electron from the atom or a MPA reaction can occur depositing more energy. The photon's energy is divided into two parts by the interaction. A portion of the energy is used to overcome the electron's binding energy and to remove it from the atom. The remaining energy is transferred to the electron as kinetic energy (photon-enhanced thermionic emission) and is deposited near the interaction site. Since the interaction creates a vacancy in one of the electron shells, typically the K or L, an electron moves down to fill in the vacancy. Even though photons are electrically neutral, they can ionize atoms indirectly through the photoelectric effect and the Compton effect. The Compton effect is a partial absorption process as the original photon has lost energy, known as Compton shift (a shift of wavelength/frequency). Either of those interactions may cause the ejection of an electron from an atom at relativistic speeds, turning that electron into a x-ray photon (secondary particle) that may ionize other atoms. Since most of the ionized atoms are due to the secondary particles, endogenous photons may also be indirectly ionizing radiation. Radiated photons are also called gamma rays if they are produced by a nuclear reaction, subatomic particle decay, or radioactive decay within the nucleus. They are called x-rays if produced outside the nucleus. The generic term “photon” is used to describe both.

The closer the electron is to the nucleus, the higher the binding energy of the shell. This is the result of the positive attraction of the protons in the nucleus. Therefore, K will have the highest energy, then L, then M and so forth. The incident electron interacts with an electron by removing it from the atom (ionization). When the target atom is ionized, it creates a hole in the electron shell. The “hole” makes the atom unstable and in an effort to stabilize itself an electron from another shell jumps down to fill the “hole”. The energy the electron must give up to jump into the hole becomes a x-ray photon. When x-ray photons are produced, they are produced isotropically (in all directions). The drop in energy of the filling electron often produces this characteristic endogenous x-ray photon. The energy of the characteristic radiation depends on the binding energy of the electrons involved. Characteristic radiation initiated by an incoming photon is referred to as fluorescent radiation. Fluorescence, in general, is a process in which some of the energy of a photon is used to create a second photon of less energy. This process sometimes converts x-rays into light photons. Whether the fluorescent radiation is in the form of light or x-rays depends on the binding energy levels in the absorbing material.

As defined herein, the linear attenuation coefficient (μ) is the actual fraction of photons interacting per 1-unit thickness of material. Linear attenuation coefficient values indicate the rate at which photons interact as they move through material and are inversely related to the average distance photons travel before interacting. The rate at which photons interact (attenuation coefficient value) is determined by the energy of the individual photons or the MPAs, and the atomic number and density of the material. This is important to the activation of the photon enhanced antimicrobial oxidizing agent according to various embodiments. In some situations, it is more desirable to express the attenuation rate in terms of the mass of the material encountered by the photons rather than in terms of distance. The quantity that affects attenuation rate is not the total mass of an object but rather the area mass. Area mass is the amount of material behind a 1-unit surface area, and is the product of material thickness and density:

Area Mass(g/cm²)=Thickness(cm)×Density(g/cm³).

The mass attenuation coefficient, using this formula, is the rate of photon interactions per 1-unit (g/cm²) area mass. According to various embodiments, by establishing a linear attenuation coefficient that does not diminish too rapidly with the functioning distance so that sufficient numbers of photons are available for enhancement of the oxidizing agent, an effective photon enhanced antimicrobial, enhanced catalyst, enhanced bleaching agent, or enhanced other described effects electronically modified oxygen derivatives, reactive oxygen species, hydrogen and its ions, oxygen and its ions, hydrons and other free radicals are generated. In various embodiments, the photon enhanced antimicrobial, catalyst, bleaching agent, or other described reactants are used in the disclosed process in plasma, liquid, gas, solid, or a combination of these states of matter. The PEOAs generated function in various embodiments of the agglomeration process disclosed herein.

Brownian diffusion is the characteristic random wiggling motion of small particles, resulting from constant bombardment by surrounding molecules. Such irregular motions of pollen grains in water were first observed by the botanist Robert Brown in 1827, and later similar phenomena were found for small smoke particles in air. In agglomeration, suspended particles tend to adhere one to the other creating bigger and heavier aggregates. The agglomeration process includes the transportation and collision of particles, and the attachment of the particles. Understanding particle agglomeration and aggregation and the mechanisms that cause such assemblies, such as diffusion, is important in a wide range of processes and applications.

As used herein, aggregation and agglomeration are two terms that are used to describe the assemblage of particles in a sample but clustering via agglomeration is irreversible. The main transport mechanisms by which particles can collide are Brownian motion, laminar or turbulent flow, or relative particle settling and gravitational agglomeration. In various embodiments, gravitational agglomeration, which is dependent on the size of the particles and their terminal velocity, is one component relating to the separation of particles in air, solutions or associated with a compound or material. Slowly settling particles interact with the more rapidly settling particles, leading to the formation of clusters. This process can be called agglomeration. Several different basic effects have been studied as being responsible for particle collision and agglomeration, which are mainly orthokinetic and hydrodynamic forces.

Brownian diffusion is instrumental in particle size selection for diffusion of photon enhanced oxidizing agent solutions created and dispersed in a fog, mist, vapor, spray, bolus, drop, stream, or other methods of dispersion.

Rates of reaction are based on collision theory. Increasing the number of collisions can lead to faster reaction rate. Increasing the concentration of reactants causes more collisions and so a faster reaction rate. Temperature increases the speed of the particles so there are more collisions and a faster reaction rate as described previously with Photon augmented oxidizing agents and photon-enhanced thermionic emission, PETE. Size of particles has an effect on solubility reactions so smaller pieces or smaller droplets have greater surface areas relative to the volume. A decrease in particle size causes an increase in the substance's total surface area when concentration remains unchanged.

Liquids evaporate only from the surface of a droplet. If the surface area of the droplet in relation to the volume is decreased, then the evaporation efficiency is increased. A substance existing in a liquid phase can be transferred to a gaseous phase by utilizing and controlling droplet size. The time needed for this phase transfer can be regulated by selecting the proper sized droplet and is a part of the designs of the displayed device.

TABLE 1

| | | TIME FOR PARTICLE |
|---|---|---|
| | DROPLET SIZE | TO FALL 10 FEET |
| FOG CLASSIFICATION | In microns | (SECONDS) |
| Wet Fog | 11-49 | 40-1,020 |
| Dry Fog | 6-10 | 1,019-12,000 |
| Extreme Dry Fog | 2-4 | 12,001-25,400 |
| Sub 2 Micron Dry Fog | <2 | >25,400 |

As shown in Table 1, the smaller the droplet size, the longer it can stay air borne. Therefore, the smaller the droplet size the faster and more efficient evaporation is achieved. According to various embodiments of the displayed device, the various micron-sized droplets evaporate at selected rates depending on application needs. In some embodiments, small size particles are selected, and they are sized so that they completely evaporate into the air before reaching most surfaces. This near 100% evaporation rate achieves near 100% chemical efficiency. In some embodiments, the particle fall rate is calculated based on density, size, and mass of the particle as well as the density of the air or gas it is placed in. Humidity also influences the fall rate outcome because at a low humidity a particle will tend to evaporate faster and lose size and mass as it remains air borne. These factors, when based on the device and methods of the embodiments, enable various embodiments of a selected size micron fog microbial suppression system, and/or agglomeration system, and/or bleaching system, or other applicable system to utilize an extremely low volume and low concentration of a photon enhanced oxidizing agent solution. These solutions generate endogenous x-ray photons that create a self-sustaining circuit of reactions. This self-sustaining circuit of reactions can be intensified by placing the photon enhanced oxidizing agent in a container or area where the endogenous x-ray photons can be reflected back into the PEOA. By reflecting these endogenous x-ray photons back into the PEOA, they are available to further ionize the PEOA solution. This device creates a PEOA solution that is more reactive and reactive longer than an oxidizing agent solution that is not enhanced with photons. This is evident by the graphs and charts included in the embodiments.

According to various embodiments, the photon enhanced oxidizing agent (PEOA) solution is deposited by the displayed device into a volume of liquid, plasma, air, or gas, or other suitable medium. In various embodiments, this is done through an existing HVAC system, a fogging device, a sprayer, a mister, an injector, a dropper, a spray can, an aerial spraying device, crop dusting, or other suitable devices. Various embodiments of the photon enhanced oxidizing agent system exhibit such a slow particle fall rate that when it is combined with the simultaneous phase change of these particles that a concentration of gas vapor (e.g., of air borne dispersion) is created and maintained of the photon enhanced oxidizing agent in the air.

The embodiments are further directed to a device and system for progressive regression of colony forming units (CFUs) from the continuous presence of a photon enhanced microbial suppression system utilizing photon enhanced oxidizing agents. Embodiments of the device and system provide a decontamination system that includes a photon enhanced microbial suppression system solution, the PEOA, and its effects on substances that it contacts. Various embodiments utilize a MPA, PETE and a photon augmented oxidizing agent containing microbial suppression device and system that includes particle size considerations for controlled dispersion and addresses agglomeration of inactivated microbes and other precipitates in a multi-faceted technology. In various embodiments of this device and system, this combination provides a means of decontaminating areas, structures, food, liquids, animals, animal fluids, plants, buildings, pipelines, homes, offices, indoors and outdoors. Some embodiments feature low chemical concentrations made more effective with the combination of the photon enhanced oxidizing agent microbial suppression device and system, so that there are reduced or no harmful effects on humans or animals or plants when administered at these low concentrations, and so exposure to the PEOA agents can be on going, constant, or nearly constant. This low concentration contrasts sharply with oxidizing agent solutions that have not been augmented with photon emissions as described. A PEOA antimicrobial solution is then applied to ambient air in a room exhibits an antimicrobial effect at concentrations at a level below published OSHA safety limits for oxidizing agent concentration in air in a habited environment. An oxidizing agent solution that has not been enhanced with photons as described would have to be applied at concentration over 100 times the allowable OSHA safety limit to achieve similar microbial reduction in the ambient air in a room. The increased efficacy results from the increased quantities of generated endogenous x-ray photons, hydrogen and its ions, oxygen and its ions, hydrons, ROS, EMODs and other free radicals in the photon enhanced oxidizing agents generated by the device and methods of the embodiments as compared to oxidizing agents that have not been exposed to photon emissions from 0.01 nm through 845 nm.

According to various embodiments, another use of the photon enhanced oxidizing agent device and system involves the dissociation of blood and other animal fluids. As a non-limiting example, blood cells contain a dramatic amount of potentially usable components such as proteins, fats, minerals, elements, and small molecular weight constituents that once separated allow disposal or repurposing of the resultant liquid in environmentally sound methods such as irrigation of crops. Animal fluids, blood, blood cells, microbes, and organic matter tend to be more difficult to dispose of as compared to serum or plasma. Blood, for example, tends to be less stable and contains total dissolved solids (TDS), total suspended solids (TSS), microbes and other components that complicate its disposal unless it is dissociated and separated. This is one of the major reasons why, for example, blood plasma (often simply referred to as plasma, i.e., an anticoagulated whole blood sample; deprived of cells and erythrocytes) and blood serum (often simply referred to as serum, i.e., coagulated whole blood; deprived of cells, erythrocytes, and most proteins of the coagulation system, especially of fibrin/fibrinogen) are considered biohazards. Various embodiments include a decontamination device and system whereby blood components go through the described agglomeration process whereby photon enhanced oxidizing agents are added to the blood causing dissociation of the blood into constituent components allowing for these components to be used for their water value and nutritional value and other desired purposes. As used herein, organic matter pertains to any carbon-based compound that exists in nature. Living things are described as organic since they are composed of organic compounds. Examples of organic compounds are carbohydrates, lipids, proteins, and nucleic acids. Since they contain carbon-based compounds, they are broken down into smaller, simpler compounds through decomposition and through dissociation when exposed to oxidizing agents that have been subject to photon emissions from 0.01 nm through 845 nm. Living organisms also excrete or secrete material that is considered an organic material. The organic matter from blood contains useful substances that have value when separated from the blood. This organic matter contains substances that can be repurposed as food sources, as fertilizer, as medicines, or other uses. According to various embodiments, the decontaminated liquid that has had particles removed through agglomeration when exposed to oxidizing agents that have been exposed to photon emissions from 0.01 nm through 845 nm will be rendered microbe free and may be used to irrigate land and/or for liquids for animals to ingest. In a period of time where water for animal ingestion is becoming a scarcer and more valuable commodity, this device and system provides a new source of nutritious water for animals and provides microbe free water for irrigation. In various embodiments, the photon emissions are a single wavelength or exist as multiple wavelengths.

TABLE 2

| | | | | | | |
|---|---|---|---|---|---|---|
| | Level Found | | Reporting | | Analyst- | Verified- |
| Analysis | As Received | Units | Limit | Method | Date | Date |

Sample ID: control Lab Number: 8948041 Date Sampled: 2021 Aug. 3

| | | | | | | |
|---|---|---|---|---|---|---|
| Nitrate-nitrogen | <0.2 | mg/L | 1.0 | EPA 300.0 | mgn8-2021 Aug. 8 | jdb5-2021 Aug. 11 |
| Biochemical oxygen demand (BOD) | 1519 | mg/L | 40 | SM 5210 B-(2011) | [symbol] m2-2021 Aug. 9 | jdb5-2021 Aug. 10 |
| Total dissolved solids | 647 | mg/L | 10 | SM 2540 C-(1997) | Mmg9-2021 Aug. 12 | mgn8-2021 Aug. 12 |
| Chemical oxygen demand (COD) | 3235 | mg/L | 500 | SM 5220 D (2011) * | [symbol] -2021 Aug. 5 | jdb5-2021 Aug. [symbol] |
| Total Kjeldahl nitrogen (TKN) | 205 | mg/L | 50.0 | PAI-DK01 * | jra [symbol] -2021 Aug. 5 | jdb5-2021 Aug. [symbol] |
| Total suspended solids | 1120 | mg/L | 4 | SM 2540 D-(2011) | Mmg9-2021 Aug. 5 | jdb5-2021 Aug. [symbol] |
| Conductivity | 2120 | μS/cm | 2 | SM 2610 B-(1997) | akn1-2021 Aug. 5 | jdb5-2021 Aug. [symbol] |
| Chloride | 157 | mg/L | 5 | EPA 300.0 | [symbol] 8-2021 Aug. 8 | jdb5-2021 Aug. 11 |

[symbol]

indicates data missing or illegible when filed

Table 2 shows testing of a wastewater sample.

TABLE 3

| | | | | | | |
|---|---|---|---|---|---|---|
| | Level Found | | Reporting | | Analyst- | Verified- |
| Analysis | As Received | Units | Limit | Method | Date | Date |

Sample ID: 12 Lab Number: 8948053 Date Sampled: 2021 Aug. 3

| | | | | | | |
|---|---|---|---|---|---|---|
| Nitrate-nitrogen [symbol] | 0.9 | mg/L | 1.0 | EPA 300.0 | ecd8-2021 Aug. [symbol] | jdb5-2021 Aug. 11 |
| [symbol] | | | | | | |
| Biochemical oxygen demand (BOD) | <15 | mg/L | 20 | SM 5210 B-(2011) | lkm2-/2021 Aug. 9 | jdb5-2021 Aug. 10 |
| Total dissolved solids | 520 | mg/L | 10 | SM 2540 C-(1997) | Mmg8-2021 Aug. 12 | jdb5-2021 Aug. 12 |
| Chemical oxygen demand (COD) | 780 | mg/L | 50 | SM 5220 D (2011) * | M [symbol] 9-2021 Aug. [symbol] | jdb5-2021 Aug. 6 |
| Total Kjeldahl nitrogen (TKN) | 148 | mg/L | 10.0 | PAI-DK01 * | j [symbol] 5-2021 Aug. [symbol] | jdb5-2021 Aug. 5 |
| Total suspended solids | 8 | mg/L | 4 | SM 2540 D-(2011) | Mmg [symbol] -2021 Aug. 5 | jdb5-2021 Aug. [symbol] |
| Conductivity | 1180 | μS/cm | 2 | SM 2510 8-(1997) | akn1-2021 Aug. [symbol] | jdb5-2021 Aug. [symbol] |
| Chloride | 159 | mg/L | 5 | EPA 300.0 | [symbol] cd8-2021 Aug. [symbol] | jdb5-2021 Aug. 11 |

[symbol]

indicates data missing or illegible when filed

Table 3 shows a test of the same wastewater as Table 1 but the device and system was used with the addition of PEOA into the wastewater. This “wastewater” now meets regulatory disposal standards for many applications.

According to various embodiments, reactions and applications de provide a multitude of uses. In some embodiments, such as HVAC applications, a low concentration of 1 part per million (ppm) of a photon enhanced oxidizing agent or even less than 1 ppm may be used to decontaminate ambient air and surfaces that are exposed to the PEOA generated by the embodiments. In other embodiments, a higher concentration of photon enhanced oxidizing agents of 50% or more may be advantageous in applications. In various embodiments, variables such as temperature, opacity of reactants, pH and others influence the selection of the concentration of oxidizing agents used by the embodiments. In some embodiments, a photon enhanced oxidizing agent, produced by the embodiments, is added to a substance (target) for antimicrobial purposes. In some embodiments, the effect of the photon emissions takes place at a certain time or place relative to the desired outcome of the reaction associated with the described methods. In these embodiments, the photon emissions will not be applied to the oxidizing agent/target mixture until such time as the photon enhanced reaction is desired to take place. In other instances, the photon emissions are applied to the oxidizing agent before it is applied to the target/mixture to be treated. An example of this is an antimicrobial and agglomeration effect in a HVAC system where applying the photon emissions to the oxidizing agent is better suited to applying the PEOA into a HVAC ductwork or blower than applying the photon emissions and oxidizing agent to the entire volume of ambient air of the HVAC system in a room or enclosure.

At present, appropriate separation/handling of animal fluids, blood, blood cells, microbes, and organic matter, e.g., by centrifugation, filtration, heating, cooling, precipitation, or analyte extraction is essential, before such processed samples can be properly and reliably disposed of or repurposed. As disclosed above, serum or plasma may be obtained from whole blood and repurposed as nutrients, fertilizer, or disposed of as needed. Cells, cell constituents, microbes, organic matter, and erythrocytes may also be removed by filtration and/or centrifugation from blood or blood components or from other animal fluids but a lower cost method is desired over present commercially available techniques. According to various embodiments, in techniques of sample processing, the animal fluids, blood, blood cells, microbes, and other organic matter of interest are first separated from the majority of substances by dissociation, agglomeration, and/or extraction methods when combined with oxidizing agents that have been exposed to photon and phonon emissions (PETE) from 0.01 nm through 845 nm. In various embodiments, extraction is performed in liquid phase or in a solid phase. In other embodiments, gross extraction of larger particles is sequenced with extraction methods processing progressively smaller units until the desired resolution is obtained. Various embodiments allow for this process to be accomplished by photon emissions applied to oxidizing agent solutions creating a PEOA. This results in an increase in ROS, EMODs, hydrogen and its ions, oxygen and its ions, beta particles, endogenous x-rays, hydrons and other free radicals when compared and contrasted with un-augmented oxidizing agents.

In various embodiments, a photon emission enhanced antimicrobial oxidizing agent solution is applied to air via a HVAC system or other suitable means. In some embodiments, a small micron (less than 20 microns droplet size) mist or fog containing photon enhanced microbial suppression system is selected to utilize an extremely low volume and low concentration of a photon enhanced antimicrobial oxidizing agent solution into a volume of air or gas. In various embodiments, a 6-10 micron droplet size, 2-4 micron droplet size, or a sub 2 micron droplet size mist or fog of a photon enhanced oxidizing agent microbial suppression system is selected. The selected droplet size is selected based on the desired fall rate of the PAOA through the ambient air. Different air qualities may be better affected by different particle sizes of PAOA.

According to various embodiments, this is done through an existing HVAC system utilizing an electrostatic fog, fogging, misting, spraying, sprinkling, diffuser, atomizer, or other suitable device. In some embodiments, the application device includes one or more of an aerosolizing nozzles producing a small micron dry fog, an air compressor to push the solution through the nozzle at the desired rate, a metering pump to dispense the solution at a rate that will give the desired concentration in ambient air, and a control system to regulate and monitor the application of the solution. In some embodiments, a small micron dry fog photon augmented oxidizing agent microbial suppression system exhibits such a slow particle fall rate that when it is combined with the simultaneous evaporation of these particles, a concentration of PEOA gas vapor is created and maintained of the photon enhanced antimicrobial agent in the ambient air serviced by the HVAC system. This can also be referred to as the target. A progressive regression of CFUs from the continuous presence of the small micron dry fog microbial suppression system provides, in the ambient air, a decontamination system of air and surfaces that the small micron dry fog microbial suppression system solution contacts. In some embodiments, as the photon enhanced antimicrobial oxidizing agent settles through the ambient air, it inactivates microbes, and any remaining PEOA in the ambient air decomposes into oxygen and water. In various embodiments, the small micron dry fog PEOA microbial suppression system is designed so that most of the microbial inactivation occurs in the HVAC system ducts and in the higher levels of a building's ambient air. By design, in various embodiment, the concentration of PEOA becomes lower as it is consumed by inactivating microbes, by evaporation, and by decomposition into oxygen and water. This demonstrates another, very different application of the technology displayed in the present disclosure. The photon enhanced EOA antimicrobial and agglomeration effects, when used in HVAC systems as described, can be modulated by utilizing x-ray reflective containers or areas where the PEOA is deployed as previously described in the methods of this invention. These x-ray reflective containers or areas allow the endogenous generated x-ray photons to remain available to react with the PEOA.

Oxidative biocides (such as chlorine and hydrogen peroxide (H₂O₂)) remove electrons from susceptible chemical groups, oxidizing them, and become themselves reduced in the process. Oxidizing agents are often low-molecular-weight compounds, and some are considered to pass easily through cell walls/membranes, whereupon they react with internal cellular components, leading to apoptotic and necrotic cell death. Although the biochemical mechanisms of action may differ between oxidative biocides, the physiological actions are largely similar. Oxidative biocides have multiple targets within a cell as well as in almost every biomolecule; these include peroxidation and disruption of membrane layers, oxidation of oxygen scavengers and thiol groups, enzyme inhibition, oxidation of nucleosides, impaired energy production, disruption of protein synthesis and, ultimately, cell death.

According to various embodiments, a generated PEOA microbial suppression system acts like a filter in that a microbial particle cannot easily pass through it without colliding with a PEOA antimicrobial particle. When a microbe collides with a PEOA antimicrobial particle, agglomeration occurs. As PEOA agglomerized microbial particles bind together, their mass increases as a unit. Gravitational forces acting on the PEOA agglomerized microbial particles increase its velocity of fall. The PEOA agglomerized microbial particles continue to gather more microbial particles as they fall through the selected medium such as liquids, air, or a gas. An analogy would be a snowball rolling downhill continually increasing in size as it advances downhill. Since PEOA antimicrobial particles contain a photon enhanced oxidizing agent, the microbe that contacts the photon enhanced oxidizing agent becomes agglomerized as it comes in contact with the PEOA antimicrobial sanitizer/disinfectant, filter particles. These agglomerized particles settle or are filtered to remove them from the solution, air, gas, liquid, or plasma.

This phenomenon is called agglomeration and solving microbial infestations with a PEOA microbial suppression particle that is generated by the device and methods displayed in this embodiment utilizes embodiments of agglomeration described in the embodiments. As used herein, agglomeration is the gathering of particle mass into a larger mass, or cluster. While this is occurring, embodiments of the photon augmented antimicrobial oxidizing agent is killing and/or deactivating the microbes. The agglomerated dead and/or deactivated microbe is pulled by gravitational forces and eventually settles from the substance being treated. In various embodiments, the substance is a liquid, gas, plasma, or any suitable substance targeted to be treated. This agglomeration of dead or inactivated microbes and other substances such as proteins and minerals is unique for a variety of reasons. As an example, in conditioned air, it has been shown that even in common air filters, such as HEPA filters designed to filter out microorganisms, arrested microorganisms can grow and, in some cases, “grow through” the filter medium and seed the air with an ever-increasing dose of microbes. Some organic media such as cellulose media provide nutrition for microbiological growth.

Various embodiments include a device that produces a progressive reduction in the microbial count as the result of the application of a PEOA enhanced antimicrobial oxidizing agent solution. This is accomplished by utilizing an antimicrobial oxidizing agent solution that has been enhanced with photons by the device and methods displayed in the embodiments to increase its effectiveness as explained previously. According to various embodiments, the wavelength of the photons utilized in this embodiment to generate PEOA is from 0.01 nm through 845 nm. In various embodiments, the wavelength of the photons is 0.01 nm through 845 nm or any combination of wavelengths in this range.

In various embodiments, one or more of trioxygen, endogenous x-rays, beta particles, hydrons, oxygen and its ions, and hydrogen and its ions are generated by the displayed device when the oxidizing agent is exposed to the photons of 0.01 nm through 845 nm and this creates a self-sustaining circuit of reactions that generates electronically modified oxygen derivatives, ROS, hydrons, hydrogen and its ions, oxygen and its ions, beta particles, x-rays and other free radicals as long as conditions allow. Various embodiments utilize hydrogen peroxide as an oxidizing agent in liquid form and ambient air as a gas. In various embodiments, the described reactions take place with reactants in different states of matter.

In an example an embodiment of a system for progressive reduction of the microbial count in ambient air, a room with 1,000,000 colony forming units (CFUs) is equipped with a HVAC system that incorporates a device and system displayed for performing the methods as disclosed herein. A small micron PEOA antimicrobial dry fog is administered into the ambient air through an existing HVAC system and device at a concentration of less than 1 part per million. This low concentration PEOA causes a reduction in the microbial CFUs as the small micron PEOA dry fog slowly settles through the air killing microbial CFUs at a rate of about 20%. After 20 minutes, the continuously administered small micron PEOA antimicrobial fog reduces the 1,000,000 CFUs by 20% to 800,000 CFUs. As the progressive regression of microbes continues, the microbial CFU count drops and after 1 hour of continuous treatment the microbial CFU count is at 512,000. With continued progressive regression, there are 262,144 CFUs of microbes in the PEOA treated air after 2 hours and 134,217 microbial CFUs after 3 hours. The ambient air continues to get cleaner and cleaner and after 5 hours the progressive regression of the microbial count with an embodiment of this PEOA system of small micron PEOA antimicrobial oxidizing agent dry fog with a photon augmented oxidizing agent has reduced the microbial CFU count to 35,184 CFUs of microbes. By continuing this PEOA reaction out for 8 hours, the microbial CFU count is reduced to 4772 CFUs. That's a 99.5% reduction in the microbial count in the PEOA treated air over an 8 hour period utilizing a progressive regression of microbes achievable with the embodiments. In contrast, independent research lab testing shows little or no microbial reduction with 1 part per million of standard hydrogen peroxide with a 5 minute contact time of the standard hydrogen peroxide with the ambient air. The same lab study shows over a 5 log reduction in microbial counts with the photon augmented oxidizing agent solution described in embodiments.

All test samples were compared against the Control provided.

Number of viable cells detected on Control Coupon=4.9×105

Number of viable cells detected on Coupon D4 (#4)=None detected. Thus, represents a 5.7 log 10 kill.

Number of viable cells detected on Coupon D8 (#8)=None detected. Thus, represents a 5.7 log 10 kill.

Independent lab testing of the HVAC antimicrobial system utilizing Photon Augmented Oxidizing Agents

Embodiments have numerous applications across many industries, from energy storage and production (displayed and illustrated in the previous photos of the PEOA fuel cell), medical, food, environmental, and others. Embodiments of the device and methods of combining photocatalytic, photochemicallytic (photochemical and photocatalytic), and dissociation reactions with photon-enhanced thermionic emission, MPA and photon augmented oxidizing agents opens a new frontier. Conventional photocatalysis decomposes oxidizing agents by disproportionation and by promoting oxidizing agent reduction instead of hydrogen liberation. Embodiments illustrate successful examples of oxidizing agent and water dissociation, wherein trioxygen associates with the reactants and suppresses the reactant reduction, thus promoting hydrogen liberation. Various embodiments of an organic photocatalytic system provide a basis of photocatalytic and photochemical and photocatalytic oxidizing agent and water dissociation. Endogenous x-ray photon production enables the photocatalytic and photochemical dissociation by freeing electrons from the atoms and molecules of the oxidizing agent and water solutions. This reaction can be further enhanced by utilizing x-ray photon reflective materials in the reaction containers or areas.

According to various embodiments, a generated PEOA microbial suppression system has had numerous applications in the petroleum industry. Hydrogen peroxide can be enhanced and ionized by the device and system and process of this disclosure so that it is extremely reactive and decomposes very rapidly giving off heat, oxygen, and water. If photon enhanced oxidizing agent (PEOA) is injected into a reservoir sand, it will decompose giving off heat and oxygen. The oxygen will then react with residual oil and organic matter generating more heat. Decomposition of PEOA is exponential with temperature increases.

Thirty percent photon enhanced hydrogen peroxide concentration will generate over 1200 BTU/lb+quality steam with about ⅓ of the heat coming from decomposition of the photon enhanced hydrogen peroxide and ⅔ coming from the reaction with formation water, paraffin and other organic substances found in the reservoir. Thus, PEOA can be used in a variety of ways to increase recovery of oil. In addition, it is well known that steam stimulation can result in formation clean-up in the vicinity of the well. Our PEOA can generate temperatures over 2000 F as it interacts with organic matter in the petroleum formation. This heat and pressure can repair formation damage, eliminate SRBs, remove paraffin, and other perform other useful actions. The released heat and the resulting increased pressure generated by our PEOA can effectively “chemically frac” an existing oil/gas well. As mentioned previously, steam stimulation from our process can clean up and open up a well with paraffin and asphalt deposits being eliminated. In addition to these effects, PEOA can generate this tremendous heat at a distance yards away from the well. As the heat and pressure buildup, formation water is vaporized, and this steam and pressure carries the PEOA further into the formation. This cycle of our PEOA being driven further and further into the formation continues until the reaction of the PEOA is depleted. This effect is modulated and controlled by regulating the amounts and rates of application of the PEOA. During treatment, heat conduction will treat the entire well bore vicinity to at least 1000 F. PEOA decomposition causes a jet stream of pressurized PEOA, hot water, steam, and heat conduction to distant areas giving 100% zonal coverage.

An oxidizing agent is a chemical species that undergoes a chemical reaction in which it gains one or more electrons. Also, an oxidizing agent can be regarded as a chemical species that transfers electronegative atoms, usually oxygen, to a substrate. An example of a common oxidizing agent is hydrogen peroxide. In the photolysis of oxidizing agents such as hydrogen peroxide and ozone, one of the oxygen-oxygen bonds in the molecule breaks. A specific quantity of energy must be added to break the bond. This is the bond energy. Data on bond energies can be obtained experimentally and is readily available on oxidizing agents. Molecular oxygen, O₂, can be photolyzed by light of 241 nm and has a bond energy of 498 kJ/mol. Hydrogen peroxide, HOOH, has a very weak O—O bond and may be photolyzed by light of 845 nm. Its bond energy is only about 142 kJ/mol. We see large difference in the strength of oxygen-oxygen bonds in these molecules due to their Lewis Structures.

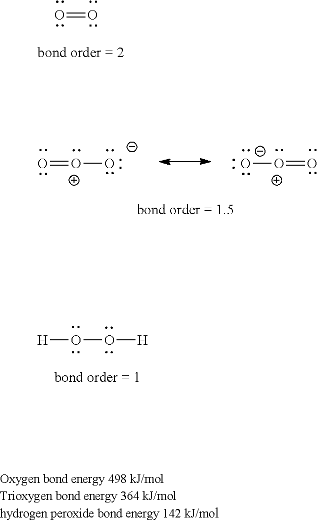

The bond energy correlates with the bond order. When bond energies are exceeded, ROS, EMODs, hydrogen and its ions, oxygen and its ions, beta particles, hydrons, x-ray photons and free radicals are released. There are various methods of meeting or exceeding these bond energies discussed herein. Bonds can be broken by exposing oxidizing agents to ionizing photons. These photons can be exogenous, but a discovery of new art displayed in this method includes the use of endogenous x-ray photons created when a target atom or molecule is ionized. This creates a hole in an electron shell. The “hole” makes the atom unstable and in an effort to stabilize itself an electron from another shell jumps down to fill the “hole”. The energy the electron must give up to move into the vacant electron hole becomes a x-ray photon. These endogenous created photons continue to break oxygen-oxygen and hydrogen to hydrogen bonds in oxidizing agents creating ROS and EMODs such as hydroxyl radicals. In readily available research, hydroxyl radicals have been shown to exist for only nanoseconds. The methods of the device and methods displayed herein demonstrate an increased and prolonged effect of the oxidizing agents that are exposed to photons of 0.01 nm through 845 nm (the upper wavelength that photolyzes oxygen to oxygen bonds in hydrogen peroxide). This prolonged and increased effect can be attributed to the prolonged and increased production of ROS, EMODs, hydrogen and its ions, oxygen and its ions, beta particle, hydrons, x-ray photons and free radicals when compared to un-augmented oxidizing agents. This effect can be modulated in the device and system by selecting a container or area that reflects the applied photons back into the generated photon enhanced oxidizing agent if desired. Since X-rays and visible light are both electromagnetic waves they propagate in space in the same way, but because of the much higher frequency and photon energy of X-rays they interact with matter very differently. Visible light is easily redirected using lenses and mirrors, but because the real part of the complex refractive index of all materials is very close to 1 for X-rays, they instead tend to initially penetrate and eventually get absorbed in most materials without changing direction. X-rays can be reflected under certain conditions when hitting matter. Mostly three reflection types are distinguished. When x-rays enter matter under grazing incidence, they will be reflected by Total External Reflection (TER) when the angle of incidence is below the critical angle. Crystal surfaces show high reflectivity under special angles depending on the wavelength of the x-rays due to Bragg-reflection. Mirrors using Bragg-reflection to redirect x-rays are called crystal mirrors. These mirrors provide large reflection angles when the reflection condition for a given wavelength is fulfilled. An x-ray mirror can be formed by fabricating a multi-layer system consisting of layers of different index of refraction. The reaction can also be modulated by selecting a container or area that reflects or scatters the endogenous created photons back into the target augmented oxidizing agent if desired. The prolonged and increased production of ROS, EMODs, hydrogen and its ions, oxygen and its ions, beta particles, hydrons, x-ray photons and free radicals can be modulated by container selection of the photon enhanced oxidizing agent. A container that allows photons to easily pass through does not reflect as many endogenous photons and as a result less endogenous x-ray photons are reflected to produce even more endogenous photons. Conversely, a container that reflects or scatters the photons back into the oxidizing agents generates a greater number of endogenous photons on a continuous basis. This amplified effect of the increased ROS, EMODs, hydrogen and its ions, oxygen and its ions, beta particles, hydrons, x-ray photons and free radicals can occur immediately when exogenous photons are applied to the oxidizing agent or an amplified effect of the increased ROS, EMODs, hydrogen and its ions, oxygen and its ions, beta particles, hydrons, x-ray photons and free radical generation can be created at a future time if the enhanced oxidizing agent is later placed in a container that reflects or scatters the endogenous photons at a later time.

Ionizing radiation consists of subatomic particles or electromagnetic waves that have sufficient energy to ionize atoms or molecules by detaching electrons from them. Gamma rays, x-rays, and the higher energy ultraviolet part of the electromagnetic spectrum are ionizing radiation, whereas the lower energy ultraviolet, visible light, nearly all types of laser light, infrared, microwaves, and radio waves are typically not thought of as ionizing radiation. Hydrogen peroxide is unique in that oxygen to oxygen bond is weak. This allows what is normally considered as non-ionizing radiation (845 nm, the upper wavelength that photolyzes oxygen to oxygen bonds in hydrogen peroxide) the ability to detach electrons from atoms or molecules. As mentioned previously, when an electron is removed from an atom or molecule and an electron from a higher orbit takes its place, energy in the form of an x-ray photon is generated and released. We refer to this as an endogenous photon.

Electromagnetic radiation can interact among themselves and with matter, giving rise to a multitude of phenomena such as reflection, refraction, scattering, polarization. X-ray photons interact with matter through the electrons contained in atoms, which are moving at speeds much slower than light. When the electromagnetic radiation (the x-rays) reaches an electron (a charged particle) it becomes a secondary source of electromagnetic radiation that scatters the incident radiation. According to the wavelength and phase relationships of the scattered radiation, we can refer to elastic processes or Compton scattering, depending on if the wavelength does not change (or changes), and to coherence (or incoherence) if the phase relations are maintained (or not maintained) over time and space. The exchanges of energy and momentum that are produced during these photon and electron interactions can even lead to the expulsion of an electron out of the atom, followed by the occupation of its energy level by electrons located in higher energy levels. Endogenous x-ray photons are generated and released in this process. In the Compton effect, the interaction is inelastic, and the radiation loses energy. This phenomenon is always present in the interaction of x-rays with matter. The incoming electrons release x-rays as they slowdown in the target (braking radiation or bremsstrahlung). The x-ray photons produced in this manner range in energy from near zero up to the energy of the electrons. An incoming electron may also collide with an atom in the target, kicking out an electron and leaving a vacancy in one of the atom's electron shells. Another electron may fill the vacancy and in so doing release an x-ray photon of a specific energy (a characteristic x-ray) which is scattered relative to the incoming electron. By scattering, we refer here to the changes of direction suffered by the incident radiation. X-ray photons scattered by atoms produce x-ray radiation in all directions, leading to interferences due to the coherent phase differences between the interatomic vectors that describe the relative position of atoms. In a molecule or in an aggregate of atoms, this effect is known as the effect of internal interference, while we refer to an external interference as the effect that occurs between molecules or aggregates. X-ray photons can be reflected off smooth metallic surfaces at very shallow angles called the grazing incidence. As a means of modulating the amount of scattered radiation, if desired, a x-ray reflecting mirror can be made of glass ceramics which is polished to give a very smooth surface (with root-mean-square surface roughness of a few Angstroms) and is coated with metal for x-ray reflection. A reflection of x-rays can occur off rougher surfaces but loss of x-ray photons through photon absorption and interaction with the reflecting surface occurs. An x-ray photon does reflect off steel, but how much depends on quite a number of factors. For example, what is the angle of the x-ray beam relative to the steel? The x-ray beam will be reflected at different intensities depending on the angle that it hits the steel. So, one can have very different intensities of reflection depending on the angle of incidence relative to the reflected x-ray photon. Another factor to consider is the distance from the x-ray photon source to the steel. The farther away, the weaker the reflection. Another factor is the wavelength (penetrating power) of the x-ray photon. Technically it can be said that a x-ray photon does not reflect, it scatters after interacting with the steel. Some x-ray photons either: penetrate completely thru the steel, are absorbed by the steel or scatter off the steel.

The energy of the x-ray photon will determine how that breaks down. In the device and methods described herein, the scattered/reflected x-ray photons can be modulated by varying the scattering/reflectiveness of the surfaces of the device container or area serviced by the device containing the photon enhanced oxidizing agent. By reflecting endogenous x-ray photons, a photon enhanced oxidizing agent can be maintained with a heightened concentration of ROS, EMODs, hydrogen and its ions, oxygen and its ions, beta particles, hydrons, x-rays and other free radicals. This heightened concentration of ROS, EMODs, Hydrogen and its ions, oxygen and its ions beta particles, hydrons, x-rays and other free radicals provides a greater oxidizing potential when compared to an oxidizing agent that has not been enhanced with photon emissions. This heightened concentration of ROS, EMODs, hydrogen and its ions, oxygen and its ions, beta particles, hydrons, x-ray photons and other free radicals provides a more effective oxidizing agent, and this heightened effectiveness is displayed in the embodiments displayed in the device and methods of the embodiments.

As used herein, an oxidizing agent can be called an oxygenation reagent or oxygen-atom transfer (OAT) agent. Oxidation reactions may involve oxygen atom transfer reactions and hydrogen atom abstraction which is a reaction where removal of an atom or group from a molecule by a radical occurs. The radiation commonly used in antimicrobial applications, photo-bleaching and other chemical processes is known as UV-C. Ultra-Violet (UV) light is invisible to the human eye and is divided into UV-A, UV-B, and UV-C. UV-C is found within 100-280 nm range. The germicidal action of UV-C is maximized at approximately 265 nm with reductions on either side. UV-C sources typically have their main emission at 254 nm. As a result, germicidal lamps can be effective in breaking down the DNA of microorganisms so that they cannot replicate and cause disease. UV radiation also can be used to eliminate trioxygen which is a Reactive Oxygen Species (ROS). Reactive nitrogen species (RNS) is a subset of reactive oxygen species Trioxygen can be used as a catalyst to convert H₂O to products that exhibit, antimicrobial properties, bleaching properties, etching properties, and other products that have wide commercial uses.

As used herein, photocatalysis is the acceleration of a photoreaction in the presence of a catalyst. Photocatalysts are materials that change the rate of a chemical reaction on exposure to light. In catalyzed photolysis, radiation is absorbed by a substrate. Photocatalytic activity (PCA) depends on the ability of the catalyst to create electron-hole pairs, which utilize electronically modified oxygen derivatives, ROS, hydrogen and its ions, oxygen and its ions, beta particles, hydrons, x-ray photons and other free radicals which are then able to undergo secondary reactions. Typically, two types of photocatalysis reactions are recognized, homogeneous photocatalysis and heterogeneous photocatalysis. As used herein, when both the photocatalyst and the reactant are in the same phase, i.e., gas, solid, or liquid, such photocatalytic reactions are termed as homogeneous photocatalysis. As used herein, when both the photocatalyst and reactant are in different phases, such photocatalytic reactions are classified as heterogeneous photocatalysis. When a photocatalyst is exposed to photon emissions of the desired wavelength (and sufficient energy), the energy of photons may be absorbed by an electron (e⁻) of valence band and it is excited to conduction band. In this process, a hole (h⁺) is created in valence band. This process leads to formation of the photo-excitation state, and a e⁻ and h⁺ pair is generated. A hydroxyl radical is generated in both types of photolysis reactions.

An example of the described reaction used as a bleaching method can be illustrated with the preparation of wood pulp as used in paper manufacturing. One of the largest volume uses of hydrogen peroxide worldwide is pulp bleaching in the paper industry. Hydrogen peroxide is also used to increase the brightness of deinked pulp. The bleaching methods are similar for mechanical pulp in which the goal is to make the fibers brighter. By using various embodiments of the device and methods and the associated reactions described herein and augmenting the hydrogen peroxide with photons with a wavelength of 0.01 nm through 845 nm, (845 nm, the upper wavelength that photolyzes oxygen to oxygen bonds in hydrogen peroxide)(0.01 nm the lower limit of x-ray photons), a synergistic reaction takes place generating ROS, EMODs, Hydrogen and its ions, beta particles, hydrons, x-ray photons, oxygen and its ions and other free radicals and also produces photo-oxidation products, photocatalytic products, and/or photochemical products by photon absorption of the oxidizing agent and target, wherein the produced photo oxidation products, photocatalytic products, and/or photochemical products cause a greater bleaching result when compared with the same concentration of un-augmented oxidizing agent in the bleaching process. The device and methods described herein allow for the same bleaching effect with a lower concentration and or volume of photon augmented oxidizing agent than un-augmented oxidizing agent. According to various embodiments, the self-sustaining circuit of reactions generated with PEOA, MPA, and PETE permits a device utilizing reactions that have not been described or utilized with a bleaching reaction previously. In addition, this reaction can be modulated by altering the x-ray photon reflectiveness of the container or area of the described reaction associated with the displayed device.

Hydrogen peroxide fuel cells have been recently described in literature. Hydrogen Peroxide may be used as an energy carrier to produce electric current. As illustrated in photos above, PEOA produces more electrical current than hydrogen peroxide that has not been exposed to photons of 0.01 nm through 845 nm. This demonstrates the functionality of our device and system.

Hydrogen peroxide is also used widely in the petroleum and petrochemical industries. An example is in the production of plastics. Propylene oxide (PO), an important bulk chemical intermediary, is used for the manufacturing of polyurethanes (polyether polyols), polyesters (propylene glycol) and solvents (propylene glycol ethers). Hydrogen peroxide dissociates generating hydroxyl radicals that react with propylene to form PO. According to various embodiment, the device and methods described in the embodiments generate more hydroxyl radicals by exposing hydrogen peroxide to photon emissions of 0.01 nm-845 nm (845 nm, the upper wavelength that photolyzes oxygen to oxygen bonds in hydrogen peroxide)(0.01 nm the lower limit of x-ray photons), wavelengths. This “enhanced” hydrogen peroxide is more reactive in generating PO then un-enhanced (standard) hydrogen peroxide due to the increase in EMODs, ROS, hydrogen and its ions, oxygen and its ions, beta particles, hydrons, endogenous x-ray photons and other free radicals produced by the methods of the embodiments. The device and system generates a self-sustaining circuit of reactions that permits an enhanced reaction that has not been described or utilized when contrasted with common reactions currently utilized in industries like the petroleum/petrochemical industries. The device's PEOA and the endogenous generated x-ray photons and endogenous generated beta particles generate a greater reactive potential by creating more ROS, EMODs, hydrogen and its ions, oxygen and its ions, beta particles, hydrons, x-ray photons and other free radicals when compared to un-augmented hydrogen peroxide that is currently used. The enhanced effect of PEOA can be modulated by altering the x-ray photon reflectiveness in containers or areas where this reaction takes place.

Hydrogen peroxide (H₂O₂) is commonly used in the dairy industry as an antimicrobial preservative. By enhancing its effectiveness with various embodiments, hydrogen peroxide exposed to photon emissions of 0.01 nm-845 nm (845 nm, the upper wavelength that photolyzes oxygen to oxygen bonds in hydrogen peroxide)(0.01 nm the lower limit of x-ray photons), generates more hydroxyl radicals and other EMODs, ROS, hydrogen and its ions, oxygen and its ions, beta particles, hydrons, and endogenous x-ray photons that exert a greater preservative and antimicrobial effect than un-enhanced H₂O₂.

Oxidizing agents are also used in the production of electronics such as microprocessors. By enhancing its effectiveness with various embodiments, hydrogen peroxide and other oxidizing agents exposed to photon emissions of 0.01 nm-845 nm (845 nm, the upper wavelength that photolyzes oxygen to oxygen bonds in hydrogen peroxide) (0.01 nm the lower limit of x-ray photons), generates more hydroxyl radicals, ROS, hydrogen and its ions, oxygen and its ions, beta particles, hydrons, endogenous x-ray photons and other EMODs. This allows for a lower concentration of H₂O₂ to provide the required quantity of ROS needed to etch circuit boards and other uses common in the electronics industry.

The list of industries that utilize oxidizing agents and the ROS/EMODs that they provide is extensive. The applications and embodiments described herein are meant to provide examples but are not meant to limit the scope of the embodiments. In addition, a partial list of oxidizing agents includes oxygen (O₂), trioxygen (O₃), hydrogen (H), hydrogen peroxide (H₂O₂), inorganic peroxides, Fenton's reagent, fluorine (F₂), chlorine (Cl₂), halogens, nitric acid (HNO₃), nitrate compounds, sulfuric acid (H₂SO₄), peroxydisulfuric acid (H₂S₂O₈), peroxymonosulfuric acid (H₂SO₅), sulfur compounds, hypochlorite, chlorite, chlorate, perchlorate, halogen compounds, chromic acid, dichromic acid, chromium trioxide, pyridinium chlorochromate (PCC), chromate, dichromate compounds, hexavalent chromium compounds, potassium permanganate (KMnO₄), sodium perborate, permanganate compounds, nitrous oxide (N₂O), nitrogen dioxide/dinitrogen tetroxide (NO₂/N₂O₄), urea, potassium nitrate (KNO₃), sodium bismuthate (NaBiO₃), ceric ammonium nitrate, ceric sulfate, cerium (IV) compounds, peracetic acid, and lead dioxide (PbO₂). This list is meant to serve as an example but is not inclusive of all oxidizing agents.

To monitor the synergistic reaction described in the embodiments, various embodiments include at least one or more sensors or other devices to indicate, detect, or inform of one or more of the following properties of the target or storage or environment: pH, oxidation and reduction potential, electrical potential, temperature, salinity, density, trioxygen concentration, oxygen concentration, hydrogen concentration, oxidizing agent concentration, flow rate, microbial content, presence or absence of bacterial species, presence or absence of corrosive metabolites or otherwise corrosive substance, identification of a gas, presence or absence of an aqueous environment, presence or absence of high, low, or otherwise concentration of bacterial or non-bacterial, biomass or non-biomass, microbial content, or location of biofilms may be used. This list is not all inclusive but is meant to provide examples of sensors and other devices that may be used singularly or in multiples. According to various embodiments, these sensors may be used to help regulate the reactions described herein.

Temperature affects reaction rate. Some of the reactions described herein are exothermic. A high pH favors hydroxyl radical formation at the expense of trioxygen formation. A low pH favors trioxygen formation over hydroxyl radical production. According to various embodiments, flow rate of the oxidizing agent as it is exposed to exogenous photon emissions is used to influence the effects of the reaction by altering the amount of time substances are exposed to the photons with the device. Also, in various embodiments, flow rate of the oxidizing agent and/or the target of the reaction described herein is used to modulate exposure to variables such as temperature, flow rate, microbes, humidity, and other conditions. This list is not inclusive but is meant as an example of effects of variables.

In some embodiments of the device and system, variables such as photon emissions dose are used to affect the generation of ROS EMODs, hydrogen and its ions, oxygen and its ions, beta particles, endogenous x-ray photons, hydrons and other free radicals. These emissions can be less than 1 second in duration if the intensity and frequency of the photon emissions is high or the time of the applied emissions can be perpetual if the dose or intensity or frequency of the emissions is low. In some embodiments, the temperature of the reaction not only affects the reaction rate but is also used to modulate enzymes present in the reactants. An example of this is the enzyme catalase. Catalase can hinder or stop reactions utilizing oxidizing agents by inactivating hydroxyl radicals. Catalase is inactivated by temperatures above certain limits. By using a sensor to measure temperature and by varying the temperature of the oxidizing agent and or the reactants enzymes such as catalase can have their effects modulated.

According to various embodiments of the device and system, the photon generating apparatus used in the methods described herein is located in or adjacent to the oxidizing agent to be enhanced. In some instances, the photon generating apparatus is located further from the oxidizing agent and methods of transmission of the photons are utilized. These methods of transmission include fiber optics, reflective materials, and other conductive media.

With an increase in temperature, there is an increase in the number of collisions between reactants. Increasing the concentration of a reactants increases the frequency of collisions between reactants and will, therefore, increase the reaction rate. An increase in temperature corresponds to an increase in the average kinetic energy of the particles in a reacting mixture—the particles move faster, colliding more frequently and with greater energy. Increasing concentration tends to also increase the reaction rate. A decrease in temperature may have the opposite effect when compared to an increase in temperature.

The rate, or speed, at which a reaction occurs depends on the frequency of successful collisions. A successful collision occurs when two reactants collide with enough energy and with the right orientation. That means if there is an increase in the number of collisions, an increase in the number of particles that have enough energy to react, and/or an increase in the number of particles with the correct orientation, the rate of reaction will increase. MPA is an example of the effects of increased collisions associated with the embodiments.

The rate of reaction is related to terms of three factors: collision frequency, collision energy, and geometric orientation. The collision frequency is dependent, among other factors, on the temperature of the reaction. When the temperature is increased, the average velocity of the particles is increased. The average kinetic energy of these particles is also increased. The result is that the particles will collide more frequently, because the particles move around faster and will encounter more reactant particles. However, this is only a minor part of the reason why the rate is increased. Just because the particles are colliding more frequently does not mean that the reaction will occur. The availability of beta particles, ionizing photons and the x-ray photon reflectivity off of containers and areas also plays an integral part in the embodiments.

Another effect of increasing the temperature is that more of the particles that collide will have the amount of energy needed to have an effective collision. In other words, more particles will have the necessary activation energy. For example, at room temperature, the hydrogen and oxygen in the atmosphere do not have sufficient energy to attain the activation energy needed to produce water:

O₂(g)+H₂(g)→No reaction

At any one moment in the atmosphere, there are many collisions occurring between these two reactants. But what we find is that water is not formed from the oxygen and hydrogen molecules colliding in the atmosphere, because the activation energy barrier is just too high, and all the collisions result in rebound. When we increase the temperature of the reactants or give them energy in some other way, the molecules have the necessary activation energy and are able to react to produce water:

O₂(g)+H₂(g)→H₂O(l)

In various embodiments, the rate of a reaction is slowed down. In some embodiments, lowering the temperature is used to decrease the number of collisions that would occur and lowering the temperature would also reduce the kinetic energy available for activation energy. If the particles have insufficient activation energy, the collisions will result in rebound rather than reaction. Using this idea, when the rate of a reaction needs to be lower, keeping the particles from having sufficient activation energy will keep the reaction at a lower rate.

In various embodiments, the humidity where the reactions described herein takes place affects the evaporation rate of the droplet if the desired location of the reaction is in the air. This variable, humidity, can change the rate of evaporation if the humidity is high and cause a decrease in the evaporation rate or cause an increase in the evaporation rate if the humidity is low in the area where the reactions described herein is to take place.

In various embodiments, where the reactions described herein take place in a liquid or gaseous environment, the opacity of the liquid or gas affects the reaction rate. In some embodiments, the more opaque a liquid or gas is, a higher dose of photons is required to achieve the desired reaction rate due to the opacity of the medium and its ability to affect interactions of the photon emissions with target materials such as oxidizing agents. Likewise, in some embodiments, the viscosity of the medium where the described reaction takes place influences the reaction rate. In some embodiments, a higher viscosity medium retards the reaction rate due to the loss of photon energy as the photons move through the medium. In other embodiments, a lower viscosity medium causes the photons to lose less energy as they move through the medium.

In some embodiments, the reactions described herein have an increased reaction rate with the addition of a catalyst. For example, iron oxides catalyze the conversion of hydrogen peroxide into oxidants capable of transforming recalcitrant contaminants. This is an example of an additive effect of a catalyst.

In some embodiments, it is desirable to slow down or halt the described reaction. For example, peroxidases or peroxide reductases are a group of enzymes which play a role in various chemical processes. They are named after the fact that they commonly break up peroxides.

Flocculants, also known as clarifying agents, are used to remove suspended solids from liquids by inducing flocculation. The solids begin to aggregate forming flakes, which either precipitate to the bottom or float to the surface of the liquid, and then they can be removed or collected. According to various embodiments of the device and system, flocculants are added to the reactions described herein before, during, or after the photon enhanced oxidizing agent is applied to the target where the described reaction is to take place. In some embodiments, flocculants are added before the reaction to remove substances that are not desired to undergo the described reaction. In other embodiments, the flocculant is added during the reaction or after the reaction depending on the desired outcome and use of the precipitated substance.

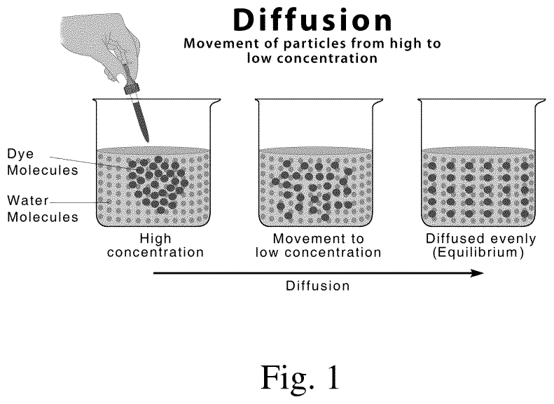

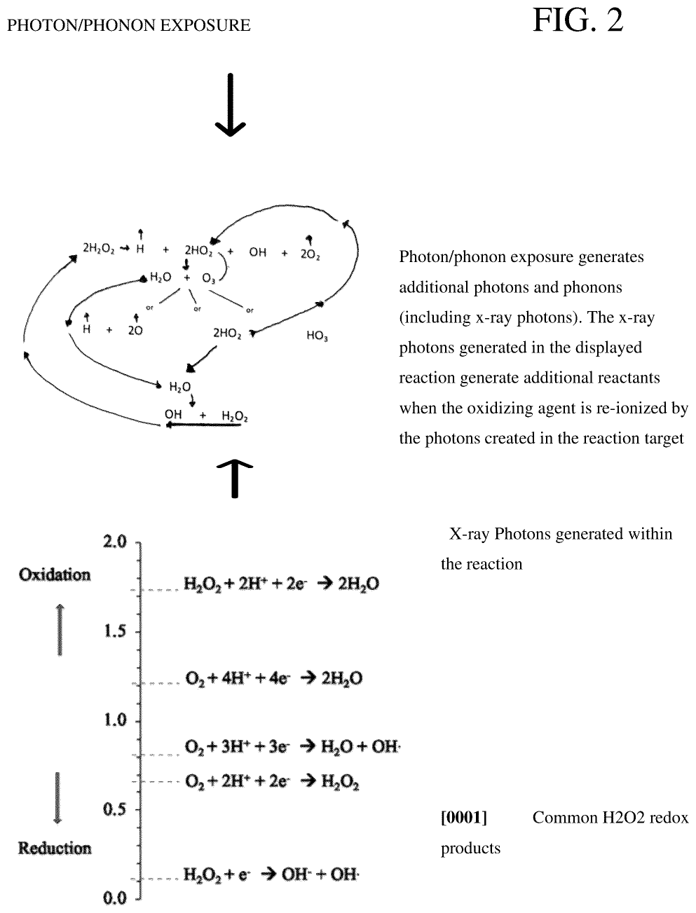

FIG. 2 is an exemplary diagram showing that a reaction can occur from a reactant molecule via an intermediate such as hydroperoxyl to form a trioxygen molecule. FIG. 1 also shows an exemplary diagram showing a “stored” oxidizing effect that can be tapped to provide reactive oxygen species, EMODs, hydrogen and its ions, oxygen and its ions, beta particles, hydrons, endogenous x-ray photons and other free radicals as needed, and the “stored” oxidizing effect feeds the self-sustained circuit of reactions so that reactive oxygen species, EMODs, hydrogen and its ions, oxygen and its ions, beta particles, hydrons, endogenous x-ray photons and other free radicals are generated until one of the reactants is depleted. During its decay back to the ground state, the trioxygen molecule created in the described reaction emits energy. This released energy provides endogenous photons and other reactants such as beta particles, electrons and hydrons to help power the continuing self-sustaining circuit of reactions.

Photons of 0.01 nm through 845 nm (845 nm, the upper wavelength that photolyzes oxygen to oxygen bonds in hydrogen peroxide) (0.01 nm the lower limit of x-ray photons), emitted on water and hydrogen peroxide creates the reaction illustrated in FIG. 2. Once the reaction is initiated, the reaction proceeds with or without further addition of photons from an outside exogenous source. The products such as ROS, beta particles, hydrons, trioxygen, hydrogen and its ions, oxygen and its ions and other generated electronically modified oxygen derivatives continue to “power” the reaction along with endogenous generated x-ray photons. An example of these products contributing to the continued reaction can be found in the decay of trioxygen. As it decays, x-ray photons are produced releasing energy to the reaction. The below photos show the increased reaction potential of the PEOA created by the device and system of the embodiments displaying the increased reaction of radiation as registered on a Geiger Counter.

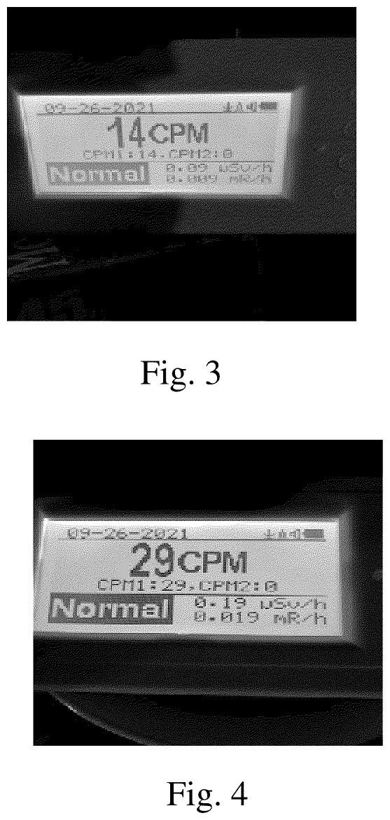

FIG. 3 records the radiation count from hydrogen peroxide that has not been exposed to photon and or phonons.

FIG. 3 shows the Geiger Counter reading of radiation from hydrogen peroxide that has been exposed to photons from 0.01 nm-845 nm (845 nm, the upper wavelength that photolyzes oxygen to oxygen bonds in hydrogen peroxide) (0.01 nm the lower limit of x-ray photons), as described by the methods in the embodiments.

The x-ray photons created by the device and methods described herein are displayed as the increased CPM count. The elevated CPM count is evident for days after the initial exogenous photon exposure of the hydrogen peroxide. Since x-ray photons are transient in nature, this sustained elevated CPM reading can only be explained by the self-sustaining circuit of reactions described in the embodiments. The elevated CPM reading displays an increase in endogenous x-ray photons from the self-sustaining circuit of reactions described in the embodiments. As discussed previously, this effect can be modulated by increasing the x-ray reflectiveness of the reaction container or area. This is evidence of the new art described by the methods herein.

FIG. 3 illustrates the enhanced effectiveness produced by an embodiment of the reactions illustrated in FIG. 2 The control substance, which is hydrogen peroxide that has not been exposed to exogenous photon emissions by the device and system as described in the embodiments, exhibited a 23.08% microbial (E. coli) reduction while the two samples of the photon enhanced solution (Sample 1 and Sample 2a) displayed a heightened effectiveness ranging between 76.92% and 84.62% microbial reduction at 4 weeks post exposure to photons of 0.01 nm through 845 nm (845 nm, the upper wavelength that photolyzes oxygen to oxygen bonds in hydrogen peroxide) (0.01 nm the lower limit of x-ray photons), This heightened residual effect supports the claim of continuing self-sustaining circuit of reactions described.

Various embodiments relate to producing one or more of trioxygen, hydrogen and/or its isotopes, oxygen and/or its isotopes, and/or electronically modified oxygen derivatives, reactive oxygen species, hydrons, free radicals, oxidizing molecules, oxygen-atom transfer (OAT) agents, oxidizing agents and/or various related species from oxidizing agents that are exposed to certain wavelengths of photon emission, exposed for certain amounts of time and exposed to certain intensities of photon emission. In various embodiments, the oxidizing agents are exposed to multiple frequencies of photon emission and multiple exposures of photon emission. In embodiments, the photons are supplied to the oxidizing agents continuously or in bursts or pulses. A continuous photon emission could be, for example, from a light emitting diode suspended in a container of an oxidizing agent emitting a constant dose of photons. Bursts or pulses of photon emission could be utilized to rapidly enhance an oxidizing agent with 0.01 nm-845 nm (845 nm, the upper wavelength that photolyzes oxygen to oxygen bonds in hydrogen peroxide) (0.01 nm the lower limit of x-ray photons), photon, for example from a high intensity laser where the high intensity bursts or pulses may be only seconds in duration, but these bursts or pulses could provide the same dose of photon emissions as a long duration continuous photon emission that was at a low dose, where dose is defined as intensity of the photon emission times the time of application.

The embodiments describe research into utilizing the effects of ionizing photons on oxidizing agents, and a discovery that offers a revolutionary and multi-disciplinary advancement to science. The disclosed device and methods provide a new paradigm to perform photocatalytic oxidation of substrates using selected photon emission as energy input, generating endogenous x-ray photons, and endogenous beta particles, trioxygen, hydrons, oxygen and its ions, and/or hydrogen and its ions as the catalysts, oxidizing agents as the oxygen source, and dissociation reactions to minimize hindrances to the reactions.

Photocatalytic activity (PCA) is commonly applied to a target where the desired reaction takes place in two distinct ways. Various embodiments utilize both methods of applying photocatalytic activity to generate unique reactions that continue even after the initial photon emissions that initiates the PCA is discontinued. As detailed in the embodiments, it has been found that the destruction of trioxygen (O₃) by certain wavelengths of photon emission prevents or retards reactions involved in the photocatalytic effects described herein. The catalyst, trioxygen, was being eliminated by certain wavelengths of photons that encourage dissociation of trioxygen. By altering the production or availability of trioxygen, according to described embodiments of the device and system, the displayed reactions include steps that allow and encourage, or alternatively prevent or retard the generation of products such as oxygen and its ions, hydrogen and its ions, hydron, reactive nitrogen species, electronically modified oxygen derivatives (EMODS), beta particles, endogenous x-ray photons and others. Some examples of EMODs are superoxide, hydrogen peroxide, hydroxyl radical, hydroxyl ion, and nitric oxide.

These EMODs are generated by exposing oxidizing agents to photons of a certain wavelength, for example between 0.01 nm and 845 nm, (845 nm, the upper wavelength that photolyzes oxygen to oxygen bonds in hydrogen peroxide) (0.01 nm the lower limit of x-ray photons), where the interaction of these agents, oxidizing agents and photons, when combined produce a total effect that is greater than the sum of the effects of the individual agents. This photon exposure from the displayed device and system generates EMODs, ROS, hydrogen and its ions, oxygen and its ions, beta particles, hydrons, endogenous x-ray photons and other free radicals. These have effects that exist for longer than typically found in nature by evidence of a residual effect created by the self-sustaining circuit of reactions which, displayed in Table 2, has shown as an increased effect that lasts for days, thereby providing a photon Enhanced Oxidizing Agent (EOA) that has increased oxidizing potential when compared with the same oxidizing agent that has not been enhanced with photons as described in the embodiments. The expected life span of EMODs ROS, hydrogen and its ions, oxygen and its ions, beta particles, hydrons, endogenous x-ray photons and other free radicals when they are found naturally in nature is measured in nanoseconds. Exposing oxidizing agents to photons as described in the embodiments produces a PEOA having a unique concentration of EMOD, ROS, hydrogen and its ions, oxygen and its ions, beta particles, hydrons, endogenous x-ray photons and other free radicals that exhibits a residual effect demonstrated by its existence for hours, days, weeks, and greater extended periods of time. As previously stated, this is evident due to the endogenous generated x-ray photons and the endogenous generated beta particles and hydrons which have previously been unreported or unrecognized. In various embodiments, the photon wavelength in a range of 0.01 nm to 845 nm (845 nm, the upper wavelength that photolyzes oxygen to oxygen bonds in hydrogen peroxide) (0.01 nm the lower limit of x-ray photons), is produced from a variety of sources such as x-ray generators, LEDs, lasers, natural light, electromagnetic radiation, arc lamps and other suitable sources. The list of radiation producing sources is not meant to limit sources to those listed but to serve as an example.

Table 4 shows actual testing results that illustrate the residual effect of the device and method generated PEOAs containing EMODs, ROS, hydrogen and its ions, oxygen its ions, beta particle, endogenous x-ray photons, hydrons and other free radicals created by embodiments of the embodiments. The test substance was a solution of 3% hydrogen peroxide, which utilized the device and methods of the embodiments, was exposed to photons to form the PEOA containing EMODs, ROS, hydrogen and its ions, oxygen and its ions, beta particles, hydrons, x-ray photons and other free radicals. The test substance or PEOA was applied to target which consisted of a microbe inoculated agar plate. The application of the PEOA can be by means such as a dropper, fog, mist or spray or any other acceptable means. This list is not meant to limit the means of application but to illustrate possible means of applications of the PEOA to the inoculum which had been placed on a carrier (agar) with a viable bacteria concentration of anaerobic bacteria Staphylococcus epidermidis ATCC 12228 A control sample consisting of an inoculated agar plate that had hydrogen peroxide that had not been enhanced with photons applied to it was also tested. PEOA was applied to inoculated plates at intervals of 1 minute, 5 minutes, 10 minutes, 30 minutes, 1 hour, 12 hours, 24 hours, 2, days, 5 days, and 7 days after radiation exposure. In one test, after 7 days, the PEOA was again subjected to photon emissions from 0.01 nm to 845 nm for the test labeled reactivation. An additional photon and or phonon exposure similar to the initial enhancement by photon and or phonons of 0.01 nm through 845 nm was the reactivation dose of photons.

TABLE 4

| | | Time After | | | | |
|---|---|---|---|---|---|---|
| | | Radiation | | | Percent | Log10 |
| | | Exposure | | | Reduction | Reduction |
| | | Substance | | | vs. | vs. |
| | Test Substance | Applied to | | | Parallel | Parallel |
| Microorganism | Concentration | Carrier | | CFU/carrier | Control | Control |
| S. epidermidis | Control | N/A | | 6.04E+05 | N/A | N/A |
| ATCC 12228 | 3% H₂O₂ | 1 | Minute | 7.00E+04 | 88.42% | 0.94 |
| | | 5 | Minute | 7.00E+04 | 88.42% | 0.94 |
| | | 10 | Minute | 3.10E+04 | 94.87% | 1.29 |
| | | 30 | Minute | 2.80E+04 | 95.37% | 1.33 |
| | | 1 | Hour | 7.10E+04 | 88.25% | 0.93 |
| | Control | 12 | Hours | 9.20E+04 | N/A | N/A |
| | 3% H₂O₂ | | | 1.80E+04 | 80.43% | 0.71 |
| | Control | 24 | Hours | 1.33E+05 | N/A | N/A |
| | 3% H₂O₂ | | | 2.10E+04 | 84.21% | 0.80 |
| | Control | 2 | Days | 3.00E+05 | N/A | N/A |
| | 3% H₂O₂ | | | 9.00E+04 | 70.00% | 0.52 |
| | Control | 5 | Days | 4.50E+04 | N/A | N/A |
| | 3% H₂O₂ | | | 3.29E+03 | 92.69% | 1.14 |
| | Control | 7 | Days | 9.80E+04 | N/A | N/A |
| | 3% H₂O₂ | | | 1.50E+04 | 84.69% | 0.82 |
| | 7 Days w/ | | | 1.00E+04 | 89.80% | 0.99 |
| | Reactivation | | | | | |

There are statistical testing variations but when comparing the increased effectiveness of the device and system generated PEOAs at 1 minute post augmentation with PEOA that was augmented 7 days previously, the results are very similar. The PEOA exhibits a pronounced residual effect. This residual effect is evidenced by the antimicrobial heightened effect of the PEOAs in reducing the microbial count. The un-enhanced oxidizing agents have been shown to exhibit an antimicrobial effect of approximately 30% at a dwell time of 5 minutes. A dose of photon exposure between 0.01 nm through 845 nm (845 nm, the upper wavelength that photolyzes oxygen to oxygen bonds in hydrogen peroxide) (0.01 nm the lower limit of x-ray photons), that has been applied to an agar plate with a known quantity of microbes has been shown to kill approximately 1% of the microbes that are exposed to it for 5 minutes. This 1% inactivation occurs solely from the 0.01 nm through 845 nm (845 nm, the upper wavelength that photolyzes oxygen to oxygen bonds in hydrogen peroxide) (0.01 nm the lower limit of x-ray photons), photon exposure. No oxidizing agent has been added. The above refers to the effects of un-enhanced oxidizing agents on microbes and contrasts sharply with the greater antimicrobial effect of PEOA. The PEOAs demonstrate an antimicrobial effect over 100% greater than un-enhanced oxidizing agents as displayed in table 3 below. The enhanced microbial reduction achieved by enhancing the oxidizing agent with a photon exposure from through 845 nm is over a 5-log reduction in the microbial count. This effect provides a concentration of a PEOA with over double the antimicrobial effect when compared to un-enhanced oxidizing agents. Also, a concentration of PEOA can be utilized that is 50% or less of the concentration of the un-enhanced oxidizing agent and exhibit the same antimicrobial activity.

Table 5 shows additional testing results that illustrate the residual effect of PEOAs containing EMODs, ROS, hydrogen and its ions, oxygen and its ions, beta particles, hydrons, endogenous x-ray photons and other free radicals created by embodiments The test substances were hydrogen peroxide at 1 ppm and at 0.3%, which were exposed or not exposed to photons of 0.01 nm through 845 nm by the embodiments (845 nm, the upper wavelength that photolyzes oxygen to oxygen bonds in hydrogen peroxide, 0.01 nm the lower limit of x-ray photons), and applied to target of microbes, which included a carrier for the inoculated target microbes such as agar with a viable bacteria concentration of Staphylococcus aureus ATCC 6538. The control for this test was a similar inoculated plate and it was treated in the same manner but there was no application of photons to the oxidizing agent.

TABLE 5

| | | | | | | Average | |
|---|---|---|---|---|---|---|---|
| | | | | | | Percent | Average [symbol] |
| | | | | | | Reduction | Reduction |
| Test | Contact | [symbol] | | | Average | Compared to | Compared to |
| Microorganisms | Time | Substance | Replicate | CFU/ml | CFU/ml | Controls | Controls |
| S. aureus | Pre- | Numbers | 1 | [symbol] | [symbol] | N/A | N/A |
| ATCC [symbol] | Treatment | Control | 2 | [symbol] | | | |
| | Post- | Numbers | 1 | [symbol] | | | |
| | Treatment | Control | 2 | [symbol] | | | |
| | 5 minutes | 1 PPM | 1 | [symbol] | [symbol] | No Reduction | No Reduction |
| | | [symbol] | 2 | [symbol] | | | |
| | | 0.5% | 1 | [symbol] | [symbol] | No Reduction | No Reduction |
| | | [symbol] | 2 | [symbol] | | | |
| | | 1 PPM | 1 | [symbol] | [symbol] | >99.9997% | [symbol] |
| | | [symbol] | 2 | [symbol] | | | |
| | | 0.5% | 1 | [symbol] | [symbol] | >99.9997% | [symbol] |
| | | [symbol] | 2 | [symbol] | | | |

[symbol]

indicates data missing or illegible when filed

In an exemplary embodiment, it is understood that after trioxygen is produced it will decay rapidly, because trioxygen is an unstable compound with a relatively short half-life. The half-life of trioxygen in liquid is shorter than in air. Trioxygen decays in liquids partly in reactions with hydroxyl radicals. The assessment of a trioxygen decay process involves the reactions of two species: trioxygen and hydroxyl radicals. Trioxygen generated by the device and methods of the embodiments decays but the reactions of produced endogenous x-ray photons and endogenous beta particles and hydrons react with the water and oxidizing agent sample to continue to generate trioxygen, ROS, EMODs, hydrogen and its ions, oxygen and its ions, beta particles, hydrons, endogenous x-ray photons and other free radicals. This continued generation of trioxygen, ROS, EMODs, hydrogen and its ions, oxygen and its ions, beta particles, hydrons, endogenous x-ray photons and other free radicals is a distinct advantage over current methods and is the reason for the extended and heightened effects of the PEOA displayed in the embodiments.

According to various embodiments, the decay of trioxygen in contact with hydroxyl radicals is characterized by a fast initial decrease of trioxygen, followed by a second phase in which trioxygen decreases by first order kinetics. In various embodiments, dependent on the composition of the liquids, the half-life of trioxygen is in the range of seconds to hours. In various embodiments, factors influencing the decomposition of trioxygen in liquids are temperature, pH, ions, cations, environment, concentrations of dissolved matter, beta particles, hydrons and photon emissions. As disclosed above, trioxygen decomposes partly in the presence of hydroxyl radicals. In various embodiments, when the pH value increases, the formation of hydroxyl radicals increases in a substance. In a solution with a high pH value, there are more hydroxide ions present, see Reaction 1 and Reaction 2. These hydroxide ions act as an initiator for the decay of trioxygen:

O₃+OH₋→HO₂₋+O₂ Reaction 1

O₃+HO₂₋→·OH+O₂·−+O₂ Reaction 2

In further exemplary embodiments, oxidative reactions due to photocatalytic, homogenous effects are described and utilized as follows:

The mechanism of hydroxyl radical production follow paths such as:

O₃+hv→O₂+O Equation 1

O+H₂O→·OH+·OH Equation 2

O+H₂O→H₂O₂ Equation 3

H₂O₂+hv→·OH+·OH Equation 4

Similarly, the Fenton system produces hydroxyl radicals by the following mechanism:

Fe²⁺+H₂O₂→HO·+Fe³⁺+OH− Equation 5

Fe³⁺+H₂O₂→Fe²⁺+HO·2+H+ Equation 6

Fe²⁺+HO·→Fe³⁺+OH− Equation 7

In photo-Fenton type processes, additional sources of OH radicals are considered: through photolysis of H₂O₂, and through reduction of Fe³⁺ ions under photon excitation:

H₂O₂+photons→HO·+HO· Equation 8

Fe³⁺+H₂O+photons→Fe²⁺+HO·+H⁺ Equation 9

Oxidative reactions due to photocatalytic heterogenous effect:

h⁺+H₂O→H⁺+·OH Equation 10

2h⁺+2H₂O→2H⁺+H₂O₂ Equation 11

H₂O₂→2·OH Equation 12

The reaction of H₂O₂=H₂O+O is typically referenced in literature as the predominant disassociation reaction associated with hydrogen peroxide and results in the production of oxygen and water. There are several reaction pathways in addition to the basic “hydrogen peroxide dissociates into water and oxygen” such as dissociation to hydronium ion and hydroperoxide, and disproportionation to dioxygen and water. Note that trioxygen is not produced in the above reactions.

According to various embodiments, trioxygen is photo-dissociated by certain wavelengths of photon emissions. In various embodiments, while trioxygen is created, it is also dissociated depending on the desired outcome of the reaction. Table 4 is a partial list of the products of trioxygen dissociation, and a partial list of the wavelengths associated with those products.

TABLE 4

| | |
|---|---|
| O(³P) + O₂(³Σ) | 1118 nm-1119 nm |
| O(³P) + O₂(¹Δ) | 599 nm-600 nm |
| O(³P) + O₂(¹Σ) | 452 nm-453 nm |
| O(¹D) + O₂(³Σ) | 402 nm-403 nm |
| O(¹D) + O₂(¹Δ) | 307 nm-308 nm |
| O(¹D) + O₂(¹Σ) | 263 nm-264 nm |
| O(³P) + O(³P) + O(³P) | 197 nm-198 nm |

According to various embodiments of the displayed device and system, in one path, the embodiments describe one or more reactions whereby the trioxygen is not totally dissociated or is partially dissociated by photon emissions. Trioxygen then becomes a photocatalyst for new reactions. In various embodiments, trioxygen is produced and retained when the wavelengths of photodissociation (e.g., Table 4) are excluded or the dose of this radiation is reduced. This exclusion or reduction coupled with photocatalytic reactions generating one or more of reactive nitrogen species, trioxygen, hydrogen and/or its isotopes, oxygen and/or its isotopes, electronically modified oxygen derivatives, reactive oxygen species, hydrons, free radicals, oxidizing molecules, oxidizing agents, beta particles, endogenous x-ray photons and/or various related species from oxidizing agents that are exposed to certain frequencies of photon emissions creates PEOA. In various embodiments, the reaction with OH— is the initial decomposition step of trioxygen decay, the stability of a trioxygen solution is thus dependent on pH and decreases as alkalinity rises. In various embodiments, at pH above 8 the initiation rate, in the presence of radical scavengers, is generally proportional to the concentrations of trioxygen and OH—. In other embodiments, in acidic solutions the reaction with OH— is not the initiation step. Predicted reaction rates below pH 4, including a mechanism based only on reaction with OH— are much lower than those determined experimentally. The trioxygen equilibrium reaction below becomes significant and the initiation reaction is catalyzed.

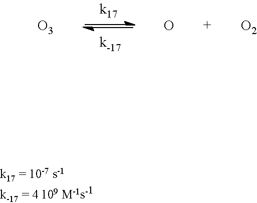

The atomic O continues to react with H₂O, or forms an excited trioxygen radical, from recombination, that subsequently reacts with H₂O, as shown in the two equations below, respectively.

O+H₂O [symbol] 2HO^{·}

O₃*+H₂O [symbol] H₂O₂+O₂

In various embodiments of the displayed device and system, the species formed then react further, forming other radicals such as O₂₋/HO₂. The propagating products, HO· and HO₂, diffuse and react with trioxygen in the continuing self-sustaining circuit of reactions that is initiated with photon emissions of 0.01 nm to 845 nm (845 nm, the upper wavelength that photolyzes oxygen to oxygen bonds in hydrogen peroxide) (0.01 nm the lower limit of x-ray photons), In addition, the endogenous x-ray photons and the endogenous beta particles and hydrons are part of the fuel that continues the self-sustaining circuit of reactions described in the embodiments.

An example of an oxidizing agent involved in this reaction: H₂O₂+photon emissions from 0.01 nm to 845 nm, (845 nm, the upper wavelength that photolyzes oxygen to oxygen bonds in hydrogen peroxide) (0.01 nm the lower limit of x-ray photons). When H₂O₂ and this selective photon emission are combined in the device and system of the displayed embodiments, this reaction yields H₂+2HO₂ which in turn yields H₂O+trioxygen. In various embodiments, this self-sustaining circuit of reactions will continue as long as the correct wavelength of exogenous or endogenous photons are present. Also, this self-sustaining circuit of reactions can proceed without the exogenous addition of photons if endogenous x-ray photons, endogenous beta particles, hydrons and other produced reactants are present and H₂O₂ (oxidizing agent) is present. In various embodiments, the two paths of this reaction yield various products but particularly H₂ and O₂ or yield 2HO₂. In some embodiments, the trioxygen that is created on this path enters and exists in this self-sustaining circuit of reactions with H₂O. The self-sustaining circuit of reactions continue to function and is partially supported by the supply of trioxygen or hydroperoxyls generated from reactions of trioxygen or hydroxyl radicals or generated from reactions of trioxygen with other reactants by the interactions of exogenous and endogenous photons and endogenous beta particles and hydrons. In various embodiments, a self-sustaining circuit of reactions includes numerous reactions and potential reactions that vary depending on variables such as temperature, pH, catalysts, and others. In the device and system of the embodiments, one of the displayed reactions in the self-sustaining circuit of reactions is exogenous and endogenous photon emissions reacting with trioxygen and water producing at various stages O2, hydroxyls, H2, HO3, HO4, hydrons and hydroperoxyls.

Exposure of oxidizing agents such as hydrogen peroxide with the entire UV spectrum of radiation produces hydroxyl radicals but limited trioxygen due to the wavelengths that are present that also destroy trioxygen. This dissociation of trioxygen was previously unappreciated and, without recognizing this and including exogenous and endogenous photon exposure, the products of this reaction will not be produced in sufficient quantities to produce a self-sustaining circuit of reactions. Furthermore, if the steps of this embodiment are performed, but performed in the wrong sequence, the reaction will not have the desired results and the self-sustaining chain of reactions will not occur. Hydroxyl radicals are very reactive free radicals, but they only exist for extremely brief periods of time measured in nanoseconds. This nanosecond long existence leads to a short-term effect whereby the hydroxyl radicals exert an influence that cannot be stored or held in reserve. Part of the uniqueness of the embodiments revolves around utilizing exogenous and endogenous photon emissions of 0.01 nm through 845 nm (845 nm, the upper wavelength that photolyzes oxygen to oxygen bonds in hydrogen peroxide) (0.01 nm the lower limit of x-ray photons), to free electrons from atoms and molecules. The ROS, EMODs, hydrogen and its ions, oxygen and its ions, beta particles, hydrons, endogenous x-ray photons and other free radicals created form a self-sustaining circuit of reactions that has an increased oxidation potential when compared to oxidizing agents that have not been exposed to the same photon emissions. The increased potential of the photon Enhanced Oxidizing Agents allows for a higher effectiveness of the oxidizing potential (as evidenced by the research studies included in the embodiments that also is evident for a period of time even after the exogenous photon emissions to the oxidizing agents have been stopped. This increased effectiveness over time is due to the endogenous x-ray photon emissions produced by the methods of this embodiment. This endogenous photon emission can be modulated by altering the x-ray photon reflectiveness in the container or in the area of the disclosed reaction. X-ray and gamma ray photons, which are at the upper end of electromagnetic spectrum, have very high frequencies and very short wavelengths. Photons in this range have high energy. They have enough energy to strip electrons from an atom or, in the case of very high-energy photons, break up the nucleus of the atom. Each ionization releases energy that is absorbed or reflected or scattered by material/matter surrounding the ionized atom. Ionizing radiation deposits a large amount of energy into a small area. In fact, the energy from one ionization is more than enough energy to disrupt the chemical bond (oxidation) between two atoms. The self-sustaining circuit of reactions displayed in this embodiment is new art which produces an increased and prolonged oxidative ability of oxidizing agent reactions that may be utilized advantageously in science and industry. This reaction is produced by the device and method of this embodiment.

According to various embodiments, the production of ROS, EMODs, hydrogen and its ions, beta particles, endogenous x-ray photons, oxygen and its ions, hydrons and other free radicals generated by the photon emissions of certain wavelengths of 0.01 nm through 845 nm (845 nm, the upper wavelength that photolyzes oxygen to oxygen bonds in hydrogen peroxide) (0.01 nm the lower limit of x-ray photons), and interaction with oxidizing agents, produces an increased concentration of reactants such as hydroperoxyls that react to form trioxygen, EMODs, ROS, hydrogen and its ions, oxygen and its ions, beta particles, hydrons, trioxidane, endogenous x-ray photons and other free radicals. With exogenous and endogenous photon emissions in a self-sustaining circuit of reactions, a steady stream of reaction products is created, one being a chain of hydroxyl radicals that can now exert a more long-lasting effect due to their continued and heightened production. In various embodiments, this self-sustaining circuit of reactions allows for a “shelf life” where the reaction is maintained and stored for future use even after the exogenous photon exposure to the oxidizing agent has been terminated. In disclosed embodiments of the displayed device and system, the increased effects and efficiencies in the oxidizing ability of the photon enhanced oxidizing agent, PEOA, that can now be measured in minutes, hours, or days due to the continued effect of the reaction products created by the self-sustaining circuit of reactions. This increased effectiveness is evident when comparing PEOA with oxidizing agents that have not been exposed to photon emissions as discussed in this embodiment.

According to various embodiments, in reference to the disclosed reactions, the embodiments described herein explain new discoveries whereby the photon emissions directed at the oxidizing agent or oxidizing agents alters the typical standard oxidation potential of oxidizing agents which is the tendency for a species to be oxidized at standard conditions. Oxidation is defined as a process in which an electron is removed from a molecule during a chemical reaction. During oxidation, there is a transfer of electrons or there is a loss of electrons.

The following embodiment of the displayed device and system relates to a working model of the equation for the self-sustaining circuit of reactions. In chemical kinetics, an equation dictates that a chemical reaction utilizing oxidizing agents proceeds via a decomposition reaction where an electron induced decomposition by photons (that may exclude wavelengths inhibiting trioxygen formation or destroying trioxygen) of the oxidizing agent proceeds. X defines potential decomposition by-products such as hydroxyls, hydroperoxyls, electronically modified oxygen species, hydrogen, oxygen, hydrons and others. In various embodiments, a reaction occurs from a reactant molecule via an intermediate such as hydroperoxyl to form a trioxygen molecule, as shown below.

OXIDIZING AGENT+photon dose(excluding wavelengths that dissociate trioxygen(O₃))→O₃+X.

In reference to the above reactions, this embodiment generates photon emissions of 0.01 nm through 845 nm (845 nm, the upper wavelength that photolyzes oxygen to oxygen bonds in hydrogen peroxide, 0.01 nm the lower limit of x-ray photons), directed at the oxidizing agent alters the typical reaction. In various embodiments, this may be accomplished by excluding wavelengths of photons that inhibit the formation of trioxygen or wavelengths that destroy trioxygen.

Photochemical reactions are a chemical reaction initiated by the absorption of energy in the form of photons. A consequence of molecules absorbing photons is the creation of transient excited states whose chemical and physical properties differ greatly from the original molecules. According to various embodiments, photochemical reactions combined with photocatalytic trioxygen generation (PTG) splits water molecules into hydrons, H₂, O₂, and O₃. PTG can achieve high dissolution in water without other competing gases found in the corona discharge method of trioxygen production, such as nitrogen gases present in ambient air. In various embodiments, this method of generation achieves consistent trioxygen concentration and is independent of air quality because water is used as the source material. Production of trioxygen photochemically was previously not utilized in reactions such as those described in the present embodiment because the required photon wavelength exclusion required to produce trioxygen as compared to producing oxygen as the typical reaction product was not understood or was underappreciated. However, as described herein, in various embodiments it is possible to change the production of oxygen by careful selection of photon wavelengths and pH such that trioxygen is preferentially produced.

Previous research involving UV radiation utilized bulbs (devices emitting electromagnetic energy) that produced a bell-shaped curve of radiation that produced wavelengths of dissociation of compounds and wavelengths creating the same compounds. While there may have been a greater influence of either the creation or dissociation wavelength, the resulting reaction was inefficient.

Thus, in various embodiments, to generate more trioxygen, photochemical reactions combined with PTG, where wavelengths of photons that dissociate trioxygen are reduced or excluded, the dose of photon emission is increased by increasing the frequency, intensity, the time the photon emission is applied, and other variables, to the dose where some or all variables may be changed to influence the result of the reaction. This demonstrates the nature of the initial complex which decomposes an oxidizing agent upon photon exposure as described in this embodiment. Further, in various embodiments, multiple reaction sequences are possible. First, comparing the electronic structure of the water and the oxidizing agent molecules, the trioxygen cleaves at least one oxygen-hydrogen bond of the water molecule in a self-sustaining circuit of reactions, which in turn, forms a hydroxyl radical plus atomic hydrogen. In various embodiments of the displayed device and system, two of the hydroxyl radicals recombine in an exoergic reaction to form an oxidizing agent molecule. The reaction reversibility dictates that upon application of trioxygen to the water molecule, the latter can decompose in one step to form oxygen atoms plus molecular hydrogen. In various embodiments, the oxygen atom in the presence of trioxygen reacts now with a water molecule by an insertion into an oxygen-hydrogen bond to form hydrogen peroxide but with the continued application of trioxygen, the generation of H₂O₂ is delayed or excluded. As the reaction is delayed, oxygen and hydrogen are liberated in sufficient quantities to alter the quantity of available components, thus preventing or minimizing the production of H₂O₂. Alternatively, in various embodiments, the oxygen atom adds itself to the oxygen atom of the water molecule forming a short-lived intermediate which then rearranges via hydrogen migration to a hydrogen peroxide molecule. The following equations display an electron induced decomposition of two water molecules in proximity (H₂O(X¹A₁))₂ to form a hydrogen peroxide molecule while liberating hydrogen and oxygen:

H₂O(X¹A₁)+TRIOXYGEN→H(²S_{1/2})+OH(X²Π_{Ω}) Equation 13

2OH(X²Π_{Ω})+TRIOXYGEN→H₂O₂(X¹A) Equation 14

H₂O₂(X¹A₁)+TRIOXYGEN→O(¹D)+H₂(X¹Σg⁺) Equation 15

O(¹D)+H₂O(X¹A₁)+TRIOXYGEN→H₂O₂(X¹A) Equation 16

O(¹D)+H₂O(X¹A₁)+TRIOXYGEN→[OOH₂(X¹A)]+TRIOXYGEN→H₂O₂(X¹A) Equation 17

(A)[(H₂O(X¹A₁))₂]+TRIOXYGEN→[H(²S_{1/2}) . . . HO(X²Π_{Ω}) . . . OH(X²Π_{Ω}) . . . H(²S_{1/2})]+TRIOXYGEN→H₂O₂(X¹A)+2H(²S_{1/2}) Equation 18

(B)[(H₂O(X¹A₁))₂]+TRIOXYGEN→[H₂(X¹Σg⁺) . . . H₂O(X¹A₁) . . . O(¹D)]+TRIOXYGEN→H₂(X¹Σg⁺)+H₂O₂(X¹A) Equation 19

(C)[(H₂O(X¹A₁))₂]+TRIOXYGEN→[H₂(X¹Σg⁺) . . . H₂O(X¹A₁) . . . O(¹D)]+TRIOXYGEN→[H₂(X¹Σg⁺) . . . H₂OO(X¹A)]+TRIOXYGEN . . . HO₃ . . . HO₄→H₂(X¹Σg⁺)+H₂O₂(X¹A) Equation 20

As can be seen above from the equations, the water solution still stores highly reactive radicals such as EMODs, hydroxyl radicals, hydroperoxyls, hydrogen and its ions, oxygen and its ions, hydrons and the like. In various embodiments, hydroxyl radicals diffuse and once they encounter a second hydroxyl radical, they recombine to form hydrogen peroxide. As described herein, it is understood that upon decomposition of water molecules, oxygen atoms are formed in a first excited state. The reactivity of ground state atoms with water is different compared to the dynamics of the trioxygen excited counterparts generated during exposure to trioxygen described in the embodiments via the stated equations.

The data and related discussion on the formation of the hydrogen peroxide molecule also explain the synthesis of atomic and molecular hydrogen during the trioxygen exposure of the oxidizing agent and/or water or solution or combination of solution composition. Here, in various embodiments of the displayed device and system, the above equations indicate that molecular hydrogen is formed in a one-step mechanism via trioxygen decomposition of the water molecule driven by the trioxygen dose generated in the solution. Alternatively, in various embodiments, the hydrogen atoms formed recombine to form molecular hydrogen. The detection of hydrogen atoms during the trioxygen exposure of the oxidizing agent, water, solution or combination of solution composition phase is a direct proof that the reactions take place. Likewise, the observation of oxygen atoms during the trioxygen exposure suggests that the reactions are also an important pathway of oxygen production. In various embodiments of the displayed device and system, the combination of photon Enhanced Oxidizing Agent and substances to be treated stores hydrogen as hydronium or other isotopes of hydrogen and as suspended “bubbles” of hydrogen even when the exogenous photon exposure is terminated and trioxygen has ceased to be produced by placing the PEOA in a sealed container so that the suspended gases are not allowed to escape. Pressure that builds due to the generated gases, in addition to the endogenous x-ray photons, maintains the reactivity and this potential can be stored for future use.

According to various embodiments, hydroxyl radicals (OH) are formed via a decomposition of a water molecule upon exposure to trioxygen. This trioxygen aided, self-sustaining circuit of reactions generates hydrogen and its ions, oxygen and its ions, x-ray photons, beta particles, hydrons and free radicals, as well as oxidizing molecules including, but not limited to, electronically modified oxygen derivatives, from water or solutions containing oxidizing agents that are exposed to photon emissions which when introduced to an effective amount of a composition containing water and/or an oxidizing agent compound or other compounds or solutions. The PEOA when combined with a target compound to be treated contains generated trioxygen, where the composition including the water and/or oxidizing agent compound, solution, or both functions together with trioxygen to lead to a reaction producing hydrogen and its ions, oxygen and its ions, electronically modified oxygen derivatives, beta particles, hydron, endogenous x-ray photons and/or solutions derived or indirectly derived. These result from the exposure of the exogenous and endogenous photon emission wavelength(s) in the self-sustaining circuit of reactions. The resultant trioxygen along with generated endogenous x-ray photons and generated beta particles used in the self-sustained circuit of reactions function in the created synergistic reaction. Also, in various embodiments there is a decomposition of the HO₂ radical to molecular oxygen plus atomic hydrogen. Finally, to generate the HO₂ radical in various embodiments of the device and system, another reaction takes place consisting of hydrogen atoms reacting with molecular oxygen. With the application by the displayed device of the correct wavelengths of photon emissions to the oxidizing agent undergoing this reaction in the self-sustained circuit of reactions, the excited state of produced hydrogen atoms and the produced molecular oxygen and the generation of trioxygen, beta particles, hydron and endogenous x-ray photons is retarded or stopped by the discontinuance of the exogenous photon emissions and the release of the created gases and endogenous photons. In various embodiments, the excited state is preserved by sealing the reactants so that produced gases are maintained and endogenous x-ray photons are reflected back into the solution, and this allows for the reactive potential to be stored.

The embodiments describe a device and system that produces a significant reaction sequence that has not been previously known, appreciated, or understood. According to various embodiments, by exposing an oxidizing agent to certain doses of photon emissions, hydrogen is liberated from the reactions described in this embodiment. In various embodiments, hydroperoxyls and trioxygen are produced when wavelengths of photon emission that dissociate trioxygen are eliminated or reduced in intensity. In another embodiments, the device and methods described in this embodiment cause the reactions to proceed when x-ray photons and beta particles and hydrons are generated and available to modulate the reaction as described. This reaction generates endogenous x-ray photons, hydrogen, oxygen, trioxygen, hydrons and other free radicals, as well as oxidizing molecules including but not limited to electronically modified oxygen derivatives. Oxidizing agents associated with the displayed device and system that are exposed to certain wavelengths of photon emissions or solutions containing oxidizing agents that are exposed to certain wavelengths of photon emissions functions together with the photon emissions of certain wavelength or wavelengths to lead to a reaction producing endogenous x-ray photons, beta particles, hydron, trioxygen, hydrogen and/or its ions, oxygen and/or its ions, electronically modified oxygen derivatives, and/or solutions derived or indirectly derived resulting from the exposure to photons of said wavelength(s) as described in this embodiment. The oxidizing potential of trioxygen is slightly less than the oxidizing potential of hydroxyl radicals, but it is greater than the oxidizing potential of hydrogen peroxide. While the commonly accepted lifetime of hydroxyl radicals is a few nanoseconds, trioxygen has been shown to maintain its reactivity for several hours. The ability of trioxygen to linger for an extended period aids the methods of this embodiment in creating a “stored” oxidizing effect. In various embodiments, the stored oxidizing effect is tapped to provide reactive oxygen species as needed and the stored oxidizing effect feeds the self-sustaining circuit of reactions so that reactive oxygen species are generated until one of the reactants is depleted.

FIG. 3 reflects testing that displays this stored oxidizing effect. When comparing the oxidizing agent control (oxidizing agent without exposure in the displayed device to photons between 0.01 nm through 845 nm) versus the photon enhanced oxidizing agent solution that has been exposed to photon emissions of 0.01 nm through 845 nm by the displayed device, (845 nm, the upper wavelength that photolyzes oxygen to oxygen bonds in hydrogen peroxide) (0.01 nm the lower limit of x-ray photons), there is over a 5-log increase in efficacy with the photon enhanced oxidizing agent solution when compared to the control see table 3. By employing the self-sustaining circuit of reactions, embodiments have increased the production of the electronically modified oxygen derivatives, ROS, hydrogen and its ions, oxygen and its ions, beta particles, hydrons, endogenous x-ray photons and other free radicals that are being continuously generated so that there are more available for use over an extended period of time due to the reactions described in this embodiment.

The above equations are exemplary and are non-limiting with respect to wavelengths, frequency, time of exposure to photon emissions, intensity of photon emissions or total dose of photon emissions associated with the displayed device and system. According to various embodiments, by exposing and utilizing the displayed device and system with the oxidizing agent or agents to photon emissions from 0.01 nm to 845 nm, (845 nm, the upper wavelength that photolyzes oxygen to oxygen bonds in hydrogen peroxide) (0.01 nm the lower limit of x-ray photons), a synergistic reaction occurs creating trioxygen and other electronically modified oxygen derivatives and disrupting the typical disassociation reaction of the oxidizing agent or agents. Chemicals such as oxidizing agents exist in a state of flux whereby, they disassociate and reassociate as self-ionization reactions occur.

When alterations of the dissociation reactions occur, new compounds or variations in compound concentrations may occur. In various embodiments of the displayed device and system, these new compounds or variations in compound concentrations created in the photon emission generated synergistic reaction generated by the displayed device enable a known oxidizing agent to create reactions that have not been observed or reported previously. In various embodiments, by restricting the photon emissions applied to the oxidizing agent so that dissociation of trioxygen is reduced or eliminated, a reaction is produced that has previously not been appreciated or reported. This is shown by the photon emissions typically produced as having wavelengths that dissociate trioxygen when said photon emission is applied to oxidizing agents. Restricting the dissociation of trioxygen has produced reaction products that have not been described for this reaction previously or that have not been produced in quantities that are shown in the present embodiments. The effect of restricting trioxygen dissociation while utilizing the endogenous generated x-ray photons has created a self-sustaining circuit of reactions that has not been previously reported.

According to various embodiments of the methods, the reactants contain enzymes, stabilizers, or other substances that affect the overall reaction rate. Enzymes, stabilizers, and/or other substances can be destroyed or inactivated by temperature variations, pH shifts, and other means. Various embodiments of these techniques are employed to arrive at favorable reaction outcomes. It is understood that phosphoric acid (H₃PO₄) is generally added to commercially available oxidizing agent solutions such as hydrogen peroxide as a stabilizer to inhibit the decomposition of the oxidizing agent. Several types of reagents, such as H₃PO₃, uric acid, Na₂CO₃, KHCO₃, barbituric acid, hippuric acid, urea, and acetanilide, have also been reported to serve as stabilizers for oxidizing agents such as hydrogen peroxide. These stabilizers have been shown to have a catalyst effect on some of the described reactions and an inhibitory effect on other areas of the reactions, but the reaction may proceed with or without stabilizers present in oxidizing agents, as desired.

Various embodiments have applications in many industries. By utilizing the displayed device and system for increasing the efficacy of oxidizing agents, common chemical reactions involving oxidizing agents are accomplished using less volume and/or a lower concentration of oxidizing agents. According to various embodiments, oxidizing agents are used to precipitate material out of solution. Increasing the efficacy of the oxidizing agent allows for this precipitation with less oxidizing agent and/or a lower concentration of oxidizing agent.

Oxidizing agents have antimicrobial properties. According to various embodiments, by increasing the antimicrobial efficacy with the device and methods described herein, concentrations of oxidizing agents utilized may be reduced while efficacy is maintained or increased. By utilizing various embodiments in a small micron antimicrobial dry fog photon enhanced oxidizing agent solution, an extremely low concentration of a photon enhanced hydrogen peroxide solution is deposited in ambient air through a HVAC system rendering the air almost microbe free in a matter of a few hours. According to various embodiments, by increasing the availability of ROS in the photon enhanced oxidizing agent solution, applications of oxidizing agents in the semiconductor industry, paper industry, petrochemical industry, and other commercial applications are accomplished faster, more economically, and/or more environmentally responsibly. The uses of the embodiments described herein are numerous and widespread in diverse industries from oil and gas to health care and beyond.

The foregoing description and accompanying figures illustrate the principles, embodiments, and modes of operation of the device and system. However, the embodiments should not be construed as being limited to the embodiments discussed herein. Additional variations of the embodiments will be appreciated by those skilled in the art. Therefore, the various embodiments should be regarded as illustrative rather than restrictive. Accordingly, it should be appreciated that variations to the embodiments described herein can be made by those skilled in the art without departing from the scope of the disclosure as defined by the following claims.

According to various embodiments, a device and method for enhancing the effectiveness of products generated from ionization reactions, photo-oxidation reactions, photocatalytic reactions, and/or photochemical reactions or a combination of these reactions is provided. The reaction products contain one or more of reactive nitrogen species, x-ray photons, hydrogen and/or its isotopes, oxygen and/or its isotopes, beta particles, hydrons, electronically modified oxygen derivatives, reactive oxygen species, trioxygen, and other free radicals. Various embodiments of the device and method include: applying at least one oxidizing agent to a target or a substance to be treated; applying photon emissions at one or more wavelength in a range of 0.01 nm through 845 nm (845 nm, the upper wavelength that photolyzes oxygen to oxygen bonds in hydrogen peroxide)(0.01 nm is the lower wavelength range for x-rays) to the oxidizing agent, the target, and/or the substance to be treated, wherein wavelengths that photo-dissociate trioxygen may be excluded; and performing an oxidizing reaction between the at least one oxidizing agent and the target and/or substance to be treated, which produces the products, and/or photochemical or a combination of these reaction products, wherein the ionization reaction products, photo oxidation reaction products, photocatalytic reaction products, and/or photochemical combined with photocatalytic reaction products generate at least one of x-ray photons, trioxygen, hydrogen and its ions, oxygen and its ions, beta particles, hydrons, hydroxyl radical, and electronically modified oxygen derivatives and other free radicals.

In various embodiments of the method, the excluded wavelengths that dissociate trioxygen are one or more of 197 nm-198 nm, 263 nm-264 nm, 307 nm-308 nm, 402 nm-403 nm, 452 nm-453 nm, 599 nm-600 nm, and 1118 nm-1119 nm.

In various embodiments, the photon emissions are applied by a photon emission source selected from an x-ray generator, electromagnetic radiation emitting bulb, a light emitting diode, an electrical ion generator or a laser or any other suitable means of generating photons of the required wavelength or wavelengths.

In various embodiments, the photon emissions are applied directly or indirectly to the oxidizing agent, and/or the target, and/or the substance or area to be treated.

In various embodiments, the at least one oxidizing agent is applied to the target or the substance or area to be treated with an oxidizing agent dispenser selected from a pump, mister, fogger, atomizer, diffuser, electrostatic sprayer, or other suitable device that dispenses the oxidizing agent in a desired particle size.

Various embodiments of the device and method further include applying additional reactants at various stages to aid the oxidizing reaction, wherein the additional reactants are selected from enzymes, catalysts, stabilizers, and flocculants or other suitable agents.

In various embodiments of the device and method, the product is used to precipitate and/or agglomerate material out of a liquid, plasma, air, or gas. In various embodiments of the device and method, the product is an antimicrobial agent. In various embodiments of the device and method, the product is a bleaching agent. In various embodiments of the device and method, the product is a catalyst, reactant, or other substance providing hydroxyl radicals, hydrogen and or its ions, oxygen and or its ions, beta particles, hydrons, EMODS, free electrons, free radicals or other reactive oxygen species.

In various embodiments of the device and method, the photon emissions are applied as a single wavelength or multiple wavelengths, applied either independently or simultaneously, and applied either continuously or pulsed. In various embodiments of the device and method, the photon emissions are applied to the oxidizing agent, the target, and/or the substance to be treated at a dose that is varied or not varied.

In various embodiments of the device and method, the amount of the at least one oxidizing agent is in a range from less than 1 part per million to 50 percent of the volume of the target and/or substance or area to be treated.

In various embodiments of the device and method, the photon emissions are applied to the at least one oxidizing agent before the at least one oxidizing agent is applied to the target and/or the substance or area to be treated, the target and/or the substance or area to be treated furthers the oxidization reaction or produces one or more additional reaction, and the further or one or more additional reactions are not dependent on continued or additional application of the exogenous photon emissions.

In various embodiments, the photon emissions are applied to the at least one oxidizing agent after the at least one oxidizing agent is applied to the target and/or substance or area to be treated so that trioxygen and other reaction products produced by the displayed device are generated after the at least one oxidizing agent is applied to the target and/or substance or area to be treated, and the oxidization reaction is readied but not initiated until a preset time or event.

In various embodiments, the oxidation reaction occurs in a sealed container whereby gases created by the oxidation reaction are not allowed to escape.

In various embodiments, x-ray photon reflective containers or areas are utilized to reflect the endogenous generated x-ray photons back into the reactants, oxidizing agents, targets, substances or areas to be treated.

In various embodiments of the method, the at least one oxidizing agent is selected from oxygen (O₂), trioxygen (O₃), hydrogen (H), hydrogen peroxide (H₂O₂), inorganic peroxides, Fenton's reagent, fluorine (F₂), chlorine (Cl₂), halogens, nitric acid (HNO₃), nitrate compounds, sulfuric acid (H₂SO₄), peroxydisulfuric acid (H₂S₂O₈), peroxymonosulfuric acid (H₂SO₅), sulfur compounds, hypochlorite, chlorite, chlorate, perchlorate, other analogous halogen compounds, chromic acid, dichromic acid, calcium oxide, chromium trioxide, pyridinium chlorochromate (PCC), chromate, dichromate compounds, hexavalent chromium compounds, potassium permanganate (KMnO₄), sodium perborate, permanganate compounds, nitrous oxide (N₂O), nitrogen dioxide/dinitrogen tetroxide (NO₂/N₂O₄), urea, potassium nitrate (KNO₃), sodium bismuthate (NaBiO₃), ceric ammonium nitrate, ceric sulfate, cerium (IV) compounds, peracetic acid, and lead dioxide (PbO₂). This list is not to be inclusive of all oxidizing agents but is meant to serve as examples of oxidizing agents.

Various embodiments of the device and method further include determining the formulation of the at least one oxidizing agent, wherein the formulation is based on one or more properties of whether the target and/or substance or area to be treated is under aerobic or anaerobic conditions, pH of the target and/or substance or area to be treated, temperature of the target and/or substance or area to be treated, salinity of the target and/or substance or area to be treated, consortium or population characteristics of organisms or micro-organism present, content of the target and/or substance or area to be treated, or content of any biofilms associated with the target and/or substance or area to be treated.

In various embodiments of the device and method, the at least one oxidizing agent further includes at least one other substance that aids in a desired process when applied to the target and/or substance or area to be treated, the desired process selected from antimicrobial properties, anti-corrosion properties, anti-neoplastic properties, thermal properties, explosive properties, precipitation properties, electrochemical properties, power generation properties or any other applicable applications of the methods of the embodiments.

In various embodiments, at least one of the photon emission wavelengths, intensity, frequency duration, or location relative to the target and/or substance or area to be treated is determined on the basis of any one or more of: the density and light absorbing or reflection or scattering quality of the target and/or substance or area to be treated; the size, shape, or composition of a container containing the target and/or substance or area to be treated; conditions or properties of the environment of the target and/or substance or area to be treated; whether the target and/or substance or area to be treated is under aerobic or anaerobic conditions; pH, temperature, salinity of the target and/or substance or area to be treated; consortium or population characteristics of any organisms or microorganisms present in the target and/or substance or area to be treated; microbial content of the target and/or substance or area to be treated; and microbial content of any biofilm present in the target and/or substance or area to be treated; or a container containing the target and/or substance or area to be treated.

In various embodiments, the concentration, temperature, viscosity, and/or pH of the at least one oxidizing agent is adjusted to produce a desired reaction or results.

In various embodiments of the device and method, the at least one oxidizing agent, target and/or substance to be treated is a liquid, solid, gas, plasma, or combination thereof, either independently or simultaneously.

In various embodiments of the device and method, the oxidation reaction is affected, inhibited, accelerated or initiated by an addition of other catalysts.

In various embodiments of the device and method, the duration of the photon emissions is in a range from less than 1 second to 30 minutes or more, the emissions continuous, pulsed, or intermittent.

In various embodiments of the device and method, the at least one oxidizing agent, target, and/or substance to be treated is heated or cooled to activate and/or inactivate enzymes present in the target and/or substance to be treated.

In various embodiments of the device and method, the pH of the oxidizing agent, target, and/or substance or area to be treated is optimized to aid in the formation of a desired reactive oxygen species, and/or wherein the pH of the oxidizing agent, target, and/or substance or area to be treated is optimized to aid in elimination or reduction in activity of selected reactive oxygen species.

According to various embodiments, a device and system is configured to perform a method for enhancing the effectiveness of products generated from ionization reactions, photo-oxidation reactions, photocatalytic reactions, photochemical reactions, and/or a combination of these reactions. The device and system includes: a reaction area, in which the at least one oxidizing agent functions together with photon emissions to perform the ionization and/or oxidation reactions, so that products of the ionization and/or oxidation reaction can be collected and separated at any time during the reactions; at least one oxidizing agent introducing component for applying the at least one oxidizing agent to the target and/or substance or area to be treated; and at least one photon emitting component for creating the photon emissions.

Various embodiments of the device and system further include one or more sensors or other devices to indicate, detect, or inform of one or more of the following properties of the reactants, target or storage or environment: pH, temperature, salinity, x-ray radiation, gamma radiation, pressure, oxidation and reduction potential, density, trioxygen concentration, oxygen concentration, hydron concentration, gamma ray concentration, beta particle concentration, hydrogen concentration, oxidizing agent concentration, flow rate, microbial content, presence or absence of bacterial species, presence or absence of corrosive metabolites or otherwise corrosive substance, identification of a gas, presence or absence of an aqueous environment, presence or absence of high, low, or otherwise concentration of bacteria or non-bacteria, biomass or non-biomass, or microbial content, and location of biofilms.

In various embodiments of the device and system, the at least one photon emitting component emits, delivers, produces, or otherwise facilitates photon emissions in a range from 0.01 nanometers to 845 nanometers, independently, simultaneous, continuously, or intermittently, and the at least one photon emitting component is suspended, adjacent to, inside of, surrounding, or associated with a container, structure, area of the at least one oxidizing agent, the target, and/or substance to be treated, and/or supported in a target container, and wherein the at least one photon emitting component is or is not physically close to the at least one oxidizing agent, the target, and/or the substance or area to be treated.

In various embodiments of the device and system, the at least one photon emitting component adjusts one or more of the photon emission wavelengths, frequency, intensity, duration, or location relative to the target and/or substance or area to be treated on the basis of any one or more of the density and light absorbing or reflection or scattering quality of the target and/or substance or area to be treated, the size, shape, or composition of the reaction area, conditions or properties of the environment, whether the target and/or substance or area to be treated is under aerobic or anaerobic conditions, pH, temperature, or salinity of the target and/or substance or area to be treated, consortium or population characteristics of any organisms or micro-organisms present in the target and/or substance or area to be treated, microbial content of the target and/or substance or area to be treated, and the microbial content of any biofilm present in the target and/or substance or area to be treated.

Inoculum Control Results

| | CHALLENGE TITER |
|---|---|
| ORGANISM | AT O HOUR(CFU/ml) |
| E. coli | 2.5 × 10⁵ |

Inoculum Test Results

| SAMPLE | TIME INTERVAL AND MICROBIAL REDUCTION | | | |
|---|---|---|---|---|
| NAME | 48 Hours | 7 Days | 14 Days | Comments |

| | | | |
|---|---|---|---|
| 8 | N = 6 | >4.14 log | >4.14 log |
| | | reduction | reduction |
| 10 | N = 20 | >4.14 log | >4.14 log |
| | | reduction | reduction |
| 3 | N = 85 | >4.14 log | >4.14 log |
| | | reduction | reduction |
| 12 | N = 66 | >4.14 log | >4.14 log |
| | | reduction | reduction |
| 15 | N = 53 | >4.14 log | >4.14 log |
| | | reduction | reduction |
| 18 | N = 74 | >4.14 log | >4.14 log |
| | | reduction | reduction |
| 25 | N = 96 | >4.14 log | >4.14 log |
| | | reduction | reduction |
| 395 | N = 120 | >4.14 log | >4.14 log |
| | | reduction | reduction |
| Jan. 11, 2021--2 | N = 31 | >4.14 log | >4.14 log |
| | | reduction | reduction |
| Jan. 11, 2021--10 | N = 91 | >4.14 log | >4.14 log |
| | | reduction | reduction |

This study was performed to determine the survival rate of various organisms when exposed to PEOA generated by the device and system as described herein. The test employed methods designed to determine antimicrobial effectiveness described in the United States Pharmacopeia.

The foregoing description and accompanying figures illustrate the principles, exemplary embodiments, and modes of operation of the invention. However, the invention should not be construed as being limited to the particular exemplary embodiments discussed above. Additional variations of the exemplary embodiments discussed above will be appreciated by those skilled in the art. Using no more than routine experimentation, one skilled in the art will recognize or be able to ascertain many equivalents to the specific embodiments and methods described herein. Such equivalents are intended to be encompassed by the scope of the following claims.

Therefore, the above-described exemplary embodiments should be regarded as illustrative rather than restrictive. Accordingly, it should be appreciated that variations to those exemplary embodiments can be made by those skilled in the art without departing from the scope of the invention as defined by the following claims. For example, the relative quantities of the ingredients may be varied to optimize the desired effects, additional ingredients may be added, and/or similar ingredients may be substituted for one or more of the ingredients described. Additional advantageous features and functionalities associated with the methods, combinations and devices of the present disclosure will be apparent from the appended claims.

What is claimed is:

1. A method for enhancing effectiveness of products generated from ionization reactions, photon-enhanced thermionic emission (PETE) reactions, multi photon absorption (MPA) reactions, photo-oxidation reactions, photocatalytic reactions, photochemical reactions, and/or a combination of these reactions, the reactions comprising one or more of oxidizing agents, hydrogen and/or its isotopes, oxygen and/or its isotopes, electronically modified oxygen derivatives, reactive oxygen species, trioxygen, beta particles, hydrons, trioxidane and other free radicals, the method comprising:
applying at least one oxidizing agent to a target or a substance or area to be treated;
applying photon emissions at one or more wavelengths in a range from 0.01 nm to 845 nm to the oxidizing agent, the target, and/or the substance or area to be treated, wherein wavelengths that photo-dissociate trioxygen may be excluded, and the photon emissions may be applied by the device to the oxidizing agent before, during, and/or after the oxidizing agent is applied to the target;
initiating and creating a reaction between the at least one photon enhanced oxidizing agent and the target and/or substance or area to be treated to produce ionization products, oxidation reaction products, reduction reaction products, photon-enhanced thermionic emission (PETE) products, multi photon absorption products, photo-oxidation reaction products, photocatalytic reaction products, photochemical reaction products, and/or a combination of these reaction products, wherein the ionization reaction products, photon-enhanced thermionic emission (PETE) products, multi photon absorption products, photo oxidation reaction products, photocatalytic reaction products, photochemical reaction products, and/or combination of these reaction products generate at least one of trioxygen, hydrogen and its ions, oxygen and its ions, hydroxyl radical, ROS, free radicals, x-ray photons, beta particles, hydrons, trioxidane, free electrons and electronically modified oxygen derivatives.

2. The method of claim 1, wherein the excluded wavelengths that dissociate trioxygen are selected from the group consisting of: 197 nm-198 nm, 263 nm-264 nm, 307 nm-308 nm, 402 nm-403 nm, 452 nm-453 nm, 599 nm-600 nm, and 1118 nm-1119 nm.

3. The method of claim 1, further comprising applying the photon emissions by an emission source or sources selected from one of an: x-ray generator, an electromagnetic radiation emitting bulb, a light emitting diode, an electrostatic charge generating device, and a laser.

4. The method of claim 1, wherein photon emissions are applied to the oxidizing agent, the target, and/or the substance or area to be treated and the emissions generate an electrostatic charge to associated particles, molecules and/or atoms.

5. The method of claim 1, further comprising applying the at least one oxidizing agent to the target, substance, or area to be treated with an oxidizing agent dispenser or dispensers with at least one of a pump, a mister, a fogger, an atomizer, a diffuser, a piezoelectric atomizer, and an electrostatic sprayer that dispenses the oxidizing agent in a desired particle size.

6. The method of claim 1, further comprising dispensing additional reactants at different intervals to aid the oxidizing reaction, wherein the additional reactants comprise at least one of enzymes, catalysts, stabilizers, ions, photons, beta particles, hydrons, reactive oxygen species, and flocculants.

7. The method of claim 1, wherein the reaction products receive an electrostatic charge and are used to precipitate and/or agglomerate material out of a liquid, plasma, air, or gas.

8. The method of claim 1, wherein the reaction products are antimicrobial agents and/or bleaching agents.

9. The method of claim 1, further comprising generating photon-enhanced thermionic emission (PETE) products and multi photon absorption products.

10. The method of claim 1, wherein the reaction products provide hydroxyl radicals, trioxidane, hydrogen and its ions, oxygen and its ions, electronically, modified oxygen derivatives (EMODS), beta particles, hydrons, free radicals and/or other reactive oxygen species.

11. The method of claim 1, further comprising adjusting viscosity of the target.

12. The method of claim 1, wherein the amount of the at least one oxidizing agent is in a range from less than 1 part per million to 50 percent or more of the volume of the target and/or substance or area to be treated.

13. The method of claim 1, further comprising applying the exogenous photon emission to the at least one oxidizing agent before, and/or after, and/or while the at least one oxidizing agent is applied to the target and/or the substance or area to be treated, the target and/or the substance or area to be treated furthers the ionization reactions and/or oxidization reactions or produces one or more additional reactions, and the further or one or more additional reactions are not dependent on continued or additional application of the exogenous photon emissions by the device, wherein the further reactions are a result of endogenous generated x-ray photons generated from the displayed device's associated reactions and the subsequently generated reactions and/or the various reaction products.

14. The method of claim 1, further comprising applying the exogenous photon emission to the at least one oxidizing agent after the at least one oxidizing agent is applied to the target and/or substance or area to be treated so that trioxygen, endogenous x-ray photons, hydrons, beta particles, hydrogen and its ions, oxygen and its ions, and trioxidane are generated after the at least one oxidizing agent is applied to the target and/or substance or area to be treated, and the ionization reaction and/or oxidization reaction is readied but not initiated until a condition is met.

15. The method of claim 1, wherein the oxidation reaction occurs in a sealed container wherein gases created by the ionization reaction and/or oxidation reaction are contained; the method further comprising reflecting or scattering generated endogenous x-ray photons by the container of the so that the generated endogenous x-ray photons are available to further ionize reactants and create a self-sustaining circuit of reactions.

16. The method of claim 1, wherein the at least one oxidizing agent comprises at least one of oxygen (O₂), trioxygen (O₃), hydrogen (H), hydrogen peroxide (H₂O₂), inorganic peroxides, Fenton's reagent, fluorine (F₂), chlorine (Cl₂), halogens, nitric acid (HNO₃), nitrate compounds, sulfuric acid (H₂SO₄), peroxydisulfuric acid (H₂S₂O₈), peroxymonosulfuric acid (H₂SO₅), sulfur compounds, hypochlorite, chlorite, chlorate, perchlorate, other analogous halogen compounds, chromic acid, dichromic acid, calcium oxide, chromium trioxide, pyridinium chlorochromate (PCC), chromate, dichromate compounds, hexavalent chromium compounds, potassium permanganate (KMnO₄), sodium perborate, permanganate compounds, nitrous oxide (N₂O), nitrogen dioxide/dinitrogen tetroxide (NO₂/N₂O₄), urea, potassium nitrate (KNO₃), sodium bismuthate (NaBiO₃), ceric ammonium nitrate, ceric sulfate, cerium (IV) compounds, peracetic acid, and lead dioxide (PbO₂).

17. The method of claim 1, further comprising determining if one or more properties of the target and/or substance or area to be treated is under aerobic or anaerobic conditions, determining pH of the target and/or substance or area to be treated, determining temperature of the target and/or substance or area to be treated, determining salinity of the target and/or substance or area to be treated, determining consortium or population characteristics of organisms or micro-organism present, determining content of the target and/or substance or area to be treated, and/or determining content of any biofilms associated with the target and/or substance or area to be treated.

18. The method of claim 1, further comprising dispersing the at least one oxidizing agent when the oxidizing agent is applied to the target and/or substance or area to be treated.

19. The method of claim 1, further comprising determining and selecting at least one of the photon emission wavelengths, frequency, intensity, duration, or location relative to the target and/or substance or area to be treated on the basis of any one or more of: density and radiation absorption, scattering or reflection quality of the target and/or substance or area to be treated; a size, shape, or composition of a container containing the target and/or substance or area to be treated; conditions or properties of the environment of the target and/or substance or area to be treated; whether the target and/or substance or area to be treated is under aerobic or anaerobic conditions; pH, temperature, salinity of the target and/or substance or area to be treated; consortium or population characteristics of any organisms or microorganisms present in the target and/or substance or area to be treated; microbial content of the target and/or substance or area to be treated; and microbial content of any biofilm present in the target and/or substance or area to be treated.

20. A system configured to perform the method claim 1, the system comprising: a target or reaction area, in which the at least one oxidizing agent functions together with photon emissions to perform the ionization reaction and/or the oxidation reaction, so that products of the ionization reaction and/or oxidation reaction can be collected and separated at any time during the reaction sequences.

21. The system of claim 20, further comprising one or more sensors configured to indicate, detect, or inform one or more properties of the target or storage or environment comprising: pH, photon emissions, pressure, temperature, salinity, density, trioxygen concentration, oxygen and oxygen ions concentration, hydrogen and hydrogen ions concentration, hydron concentration, oxidizing agent concentration, flow rate, microbial content, mass, oxidation or reduction potential, electrical potential, presence of ionizing radiation, presence or absence of bacterial species, presence or absence of corrosive metabolites or otherwise corrosive substance, identification of a gas, presence or absence of an aqueous environment, presence or absence of high, low, or otherwise concentration of bacteria or non-bacteria, biomass or non-biomass, or microbial content, and location of biofilms.

22. The system of claim 20, further comprising at least one photon emitting component, wherein the at least one photon emitting component has photon emissions from 0.01 nanometers to 845 nanometers.

23. The system of claim 22, wherein the at least one photon emitting component adjusts one or more of the generated photon emission wavelengths, frequency, intensity, duration, or location relative to the target and/or substance or area to be treated on the basis of one or more of the density and light transmission potential of the target.

24. The method of claim 1, wherein concentration, temperature, viscosity, and/or pH of the at least one oxidizing agent are adjusted or modulated by the device to produce a desired reaction or results.

25. The method of claim 1, further comprising affecting or initiating the ionization and/or oxidation reaction by adding of photon emissions of from 0.01 nm through 845 nm.

26. The method of claim 1, wherein the duration of the device generated photon emissions is in a range from 1 second to 30 minutes.

27. The method of claim 1, further comprising applying heating or cooling to modulate the reaction.

28. The method of claim 1, wherein the pH of the oxidizing agent, target, and/or substance or area to be treated is optimized by the device to aid in the formation of a desired reactive oxygen species and/or wherein the pH of the oxidizing agent, target and/or substance or area to be treated is optimized by the device to aid in elimination or reduction in activity of selected reactive oxygen species.
